(19) United States
(12) Patent Application Publication
(10) Pub. No.: US 2024/0239903 A1
(43) Pub. Date: Jul. 18, 2024

(54) PATCHED-1 MONOCLONAL ANTIBODIES AND METHODS OF USE

(71) Applicant: THE BOARD OF REGENTS OF THE UNIVERSITY OF TEXAS SYSTEM, Austin, TX (US)

(72) Inventors: Linda DONNELLY, Arlington, TX (US); Xiaofeng QI, Dallas, TX (US); Xiaochun LI, Dallas, TX (US)

(73) Assignee: THE BOARD OF REGENTS OF THE UNIVERSITY OF TEXAS SYSTEM, Austin, TX (US)

(21) Appl. No.: 18/409,571

(22) Filed: Jan. 10, 2024

Related U.S. Application Data
(60) Provisional application No. 63/438,948, filed on Jan. 13, 2023.

(51) Int. Cl. C07K 16/28; A61K 45/06; G01N 33/68

(52) U.S. Cl. CPC C07K 16/2863; A61K 45/06; G01N 33/6893; C07K 2317/21; C07K 2317/24; C07K 2317/76; G01N 2333/705; G01N 2800/7057

(57) ABSTRACT

Disclosures herein are directed to antibodies and epitope binding agents that specifically bind to the extracellular domain of Patched-1 and compositions thereof for use in clinical and non-clinical applications. Also provided are methods to treat and/or prevent Hedgehog pathway related disorders.

### CROSS REFERENCE TO RELATED APPLICATION

This application claims priority to U.S. Provisional Patent Application Ser. No. 63/438,948, filed Jan. 13, 2023, and titled “PATCHED-1 MONOCLONAL ANTIBODIES AND METHODS OF USE,” which is incorporated by reference herein in its entirety.

### ACKNOWLEDGEMENT OF GOVERNMENT SUPPORT

This invention was made with government support under Grant Nos. HL020948 and GM135343 awarded by the National Institutes of Health. The government has certain rights in the invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 31500 bytes in size and titled “106546-783768.xml.”

### BACKGROUND

### 1. Field

The present disclosure is directed to antibodies that recognize structural features of the extracellular domain of folded human PATCHED-1 protein, compositions comprising the antibodies, and methods of using the compositions.

### 2. Discussion of Related Art

The Hedgehog (Hh) signaling pathway is an important regulatory mechanism that plays a critical role during embryonic pattern formation and differentiation in vertebrates. Hh signaling is known to regulate the proliferation of neuronal progenitor cells, such as cerebella granule cells and neural stem cells. In the developing intestinal tract, a low-level of Hh signaling is required for pancreatic development, while a high-level of Hh signaling blocks pancreatic organogenesis. Hh is also known to be critical in stem cell proliferation and organogenesis in skin, prostate, testis and bone marrow. Several cell-cycle and proliferation regulatory genes, such as c-myc and cyclin D and E are among the target genes of Hh signaling. The pathway has also been implicated in tumorigenesis and development of some cancers. The Hh signaling pathway is mediated by Hh ligands including Sonic Hedgehog (Shh), Indian Hedgehog (Ihh) and Desert Hedgehog (Dhh). Constitutive activation or deregulation of Hh pathway has been implicated to cause of range of Hh-related disorders including neuronal proliferative diseases, tumorigenesis in BCC, medulloblastoma (the most common childhood brain tumor), rhabdomyosarcoma, pancreatic cancer, small cell lung cancer, prostate cancer, breast cancer, benign prostate hyperplasia, wet macular degeneration, psoriasis, bone marrow proliferative diseases, osteopetrosis and leukemias.

Protein patched homolog 1 (PTCH1 or Patched-1) protein is a receptor for the secreted Hedgehog ligands including sonic hedgehog. It is a 12-pass transmembrane receptor and acts as a negative regulator of Hh signaling. On binding to the ligand, Patched-1 is trafficked away and relieves inhibition of G-protein coupled receptor smoothened, that results in downstream signaling causing cell proliferation. Patched-1 is known to function as a tumor suppressor. Mutations in the PTCH1 gene, that encodes Patched-1 are linked to nevoid basal cell carcinoma syndrome esophageal squamous cell carcinoma, trichoepitheliomas, transitional cell carcinomas of the bladder, as well as holoprosencephaly. Loss-of-function mutations of PTCH1 are found in Gorlin's syndrome (a hereditary syndrome with high risk of skin and brain cancers, also known as Basal Cell Nevus Syndrome (BCNS)).

To date there are no commercially available antibodies that recognize the folded Patched-1 from human cells and successfully compete with Hedgehog for binding to Patched-1. Such antibodies could be useful in a wide range of clinical and non-clinical applications. There is therefore a need in the field for robust highly sensitive Patched-1 antibodies that can be used for diagnostic and therapeutic purposes.

### SUMMARY

In some aspects, the current disclosure encompasses an isolated antibody, epitope binding fragment, or variant thereof that binds to Patched-1, comprising: an immunoglobulin heavy chain variable region (V_{H}) that comprises at least 80% identity to the amino acid sequence set forth in SEQ ID NO: 1; and/or an immunoglobulin light chain variable region (V_{L}) that comprises at least 80% identity to the amino acid sequence set forth in SEQ ID NO: 2. In some aspects, the isolated antibody, epitope binding fragment, or variant thereof comprises an immunoglobulin heavy chain variable region (V_{H}) that comprises the amino acid sequence set forth in SEQ ID NOs: 1, 3 or 5; and/or an immunoglobulin light chain variable region (V_{L}) that comprises the amino acid sequence set forth in SEQ ID NOs: 2, 4 or 6.

In some aspects, the isolated antibody, epitope binding fragment or variant thereof as disclosed herein comprises one or more of: HC-CDR1 comprising the amino acid sequence at least 60% identical to the amino acid sequence set forth of SEQ ID NO: 7, an HC-CDR2 comprising the amino acid sequence at least 60% identical to the amino acid sequence set forth of SEQ ID NO: 8 or SEQ ID NO: 9; an HC-CDR3 comprising the amino acid sequence at least 60% identical to the amino acid sequence set forth of SEQ ID NO: 10; an LC-CDR1 comprising the amino acid sequence at least 60% identical to the amino acid sequence set forth of SEQ ID NO: 11 or SEQ ID NO: 12; an LC-CDR2 comprising the amino acid sequence at least 60% identical to the amino acid sequence set forth of SEQ ID NO: 13 or SEQ ID NO: 14; and an LC-CDR3 comprising the amino acid sequence of any one of SEQ ID NO: 15.

In some aspects, the isolated antibody, epitope binding fragment or variant thereof comprises a V_{H} comprising the amino acid sequence comprising at least 80% identity to the amino acid sequence set forth in any one of SEQ ID NOs: 7-10. In some aspects, the isolated antibody, epitope binding fragment or variant thereof comprises a V_{L} comprising the amino acid sequence at least 80% identity to the amino acid sequence set forth in any one of SEQ ID NOs: 11-15.

In some aspects, the isolated antibody, epitope binding fragment or variant thereof comprises any one of: a variable region having the sequence of SEQ ID NO: 1 and a light chain variable region having the sequence of SEQ ID NO: 2, a heavy chain variable region having the sequence of SEQ ID NO: 1 and a light chain variable region having the sequence of SEQ ID NO: 4, a heavy chain variable region having the sequence of SEQ ID NO: 1 and a light chain variable region having the sequence of SEQ ID NO: 6, a heavy chain variable region having the sequence of SEQ ID NO: 3 and a light chain variable region having the sequence of SEQ ID NO: 2, a heavy chain variable region having the sequence of SEQ ID NO: 3 and a light chain variable region having the sequence of SEQ ID NO: 4, a heavy chain variable region having the sequence of SEQ ID NO: 3 and a light chain variable region having the sequence of SEQ ID NO: 6, a heavy chain variable region having the sequence of SEQ ID NO: 5 and a light chain variable region having the sequence of SEQ ID NO: 2, a heavy chain variable region having the sequence of SEQ ID NO: 5 and a light chain variable region having the sequence of SEQ ID NO: 4, or a heavy chain variable region having the sequence of SEQ ID NO: 5 and a light chain variable region having the sequence of SEQ ID NO: 6.

In some aspects, the isolated antibody, epitope binding fragment or variant thereof of is a full-length antibody. In some aspects, the isolated antibody, epitope binding fragment or variant thereof is for example a monoclonal antibody, an IgG, Fv, single chain antibody, nanobody, diabody, scFv, Fab, F(ab′)2, and Fab. In some aspects, the isolated antibody, epitope binding fragment or variant thereof is a human or a humanized antibody. In some aspects, the isolated antibody, epitope binding fragment or variant thereof further comprises a detection molecule. Non-limiting examples of detection molecule include a fluorescent label, phosphorescent molecules, chemiluminescent molecules, radioactive isotope, chromophores, luminescent molecules, photoaffinity molecules, colored particles and/or ligands, such as biotin fluorescent dyes, electrochemiluminescence dyes, metal-chelate complexes or labels.

In some aspects, the current disclosure also encompasses a pharmaceutical composition, comprising the isolated antibody, epitope binding fragment or variant thereof as disclosed herein and a pharmaceutically acceptable carrier. In some aspects, the pharmaceutical composition of may further comprise additional therapeutic agents for example, a chemotherapeutic. In some aspects, the pharmaceutically acceptable excipient comprises one of more of a buffer, diluent, excipient, filler and/or an encapsulating material.

In some aspects, the current disclosure also encompasses a polynucleotide sequence encoding an isolated antibody, epitope binding fragment or variant thereof as provided herein. In some aspects the polynucleotide comprises a nucleic acid sequence at least 60% identical to one or more of an nHC-CDR1 corresponding to any one of SEQ ID NOs: 16 or 17, an nHC-CDR2 corresponding to any one of SEQ ID NOs: 18 or 19, an nHC-CDR3 corresponding to any one of SEQ ID NOs: 20, 21, or 20, an nLC-CDR1 corresponding to any one of SEQ ID NOs: 22, 23, or 24, nLC-CDR2 corresponding to any one of SEQ ID NOs: 25 or 26, and an nLC-CDR3 corresponding to any one of SEQ ID NO: 27. In some aspects, the polynucleotide encodes a variable heavy chain (VA) comprising a nucleic acid sequence with at least 80% identity to any one of SEQ ID NOs: 28, 29, or 30. In some aspects, the polynucleotide disclosed herein, encodes a variable light chain (V_{L}) comprising a nucleic acid sequence with at least 80% identity to of SEQ ID NOs: 31, 32, or 33.

In some aspects, the current disclosure also encompasses a host cell comprising the polynucleotide as disclosed herein. In some aspects, the host cell comprises a first nucleic acid encoding any one of the variable heavy chain (V_{H}) polypeptide provided herein; and/or a second nucleic acid encoding any one of the variable light chain (V_{L}) polypeptide provided herein. In some aspects, the host cell is a splenic B lymphocyte or a hybridoma.

In some aspects, the current disclosure also encompasses a method of isolating the antibody, or epitope binding fragment as provided herein, the method comprising culturing the host cell provided herein under conditions suitable for the cell to express the antibody, epitope binding fragment and purifying the antibody or epitope binding fragment from the cell. In some aspects, the current disclosure also encompasses a method of treatment or prophylaxis in a subject suffering from or suspected of hedgehog pathway related disease, the method comprising administering to the subject an effective amount of the pharmaceutical composition disclosed herein. In some aspects, examples of Hh related conditions include cancer, pulmonary disease, neuronal disease or an inflammatory disease. In some aspects, the subject is a human.

In some aspects, the current disclosure also encompasses a method of detecting Patched-1 on the surface of a cell, the method comprising: (a) contacting the cell with the isolated antibody, epitope binding fragment or variant thereof of any one of claims 1-10; wherein the isolated antibody, epitope binding fragment or variant thereof specifically binds to Patched-1 to comprise an antibody-Patched-1 complex; (b) using a detector to determine presence of the antibody-Patched-1 complex; and (c) quantifying the levels of Patched-1 on the cell. In some aspects, the method further comprises a reporter molecule that specifically binds to the isolated antibody, epitope binding fragment or variant thereof. Non-limiting examples of suitable detectors include detector is selected from a microscope, a FACS, fluorescent detector, chemiluminescent detector, autoradiography equipment, or a scintillation counter.

In some aspects, the current disclosure also encompasses a kit comprising: an isolated antibody, epitope binding fragment or variant thereof as disclosed herein, a reporter molecule that detects, reagents and instructions for use. In some aspects, the current disclosure also encompasses a kit for IHC and related applications comprising an isolated antibody, epitope binding fragment or variant thereof as disclosed herein, reagents and instructions for use.

### BRIEF DESCRIPTION OF THE DRAWINGS

The following drawings form part of the present specification and are included to further demonstrate certain aspects of the present disclosure, which can be better understood by reference to the drawing in combination with the detailed description of specific aspects presented herein. Aspects of the present inventive concept are illustrated by way of example in which like reference numerals indicate similar elements and in which:

FIG. 1 shows pull down assay of antibodies and Hh-N by Patched-1.

FIG. 2A provides a Cryo-EM structure of Patched-1-6H3^{Fab} complex.

FIG. 2B provides a structural comparison showing the steric conflicts (indicated by dashed ovals) between 6H3 and Hh-N. The TMs, ECD1, ECD2 of Patched-1, 6H3 and Hh-N are shown in blue, green, pink, magenta and orange, respectively. The interfaces between Patched-1 and Hh-N are indicated by arrows.

FIG. 3 is a graph showing data from a GLI1-dependent Luciferase assay: Abs and HH conditioned medium were added to HH-Light II cells. Data are shown as mean±SD.

FIG. 4 shows in vivo assay in mice. 7-8 weeks old female mice were shaved, and the bottom half of dorsal region were depilated with Nair. 6H3^{Fab} was injected (I.P., 20 mg/g mice) every other day for 14 days. The photos were taken on Day 14.

FIG. 5 Skin tissues were harvest on Day 14 and the mRNA levels of Gli1 were detected by RT-PCR. GAPDH was used as an internal control. Data were processed using ΔΔCt method and shown as mean±SD.*, p<0.05, t-test.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The following detailed description references the accompanying drawings that illustrate various aspects of the present inventive concept. The drawings and description are intended to describe aspects and aspects of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, in part, on the identification of a panel of mouse monoclonal antibodies that specifically target the folded extracellular domain of Patched-1. Patched-1 is a transmembrane receptor that binds to Hh ligands and plays a critical role in regulating the Hh signaling pathway. Accordingly, in some aspects, the current disclosure provides epitope binding agents, for example an antibody or a fragment/variant/conjugate thereof, that specifically binds to folded Patched-1. In some aspects, the current disclosure encompasses compositions, for example pharmaceutical compositions, comprising these epitope binding agents. In some aspects, the current disclosure also provides methods of making these epitope binding agents and compositions thereof. In some aspects, the current disclosure also provides methods of using the compositions provided herein in clinical and non-clinical applications.

### I. Terminology

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues see, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by the amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

The term “antibody” herein is used in the broadest sense and encompasses various antibody structures, including but not limited to monoclonal antibodies, polyclonal antibodies, recombinant antibody, single domain antibodies, nanobodies, multispecific antibodies (e.g., bispecific antibodies), and antibody fragments so long as they exhibit the desired antigen-binding activity. The term is further defined and elaborated on in the detailed description.

An “antibody fragment” or “epitope binding fragment” or “antigen binding fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv); and multispecific antibodies formed from antibody fragments. A part or fragment of the antibody may correspond to at least 1%, at least 2%, at least 3%, at least 4%, at least 5%, at least 10%, at least 20%, at least 30%, at least 40% of the length of the disclosed sequence, such as represented by an amino acid sequence with a specific SEQ ID NO, or at least 50%, or at least 60%, or at least 70%, or at least 80%, or at least 90% of the length.

An “epitope binding agent” comprises an antibody, or epitope binding fragment or a variant and/or conjugate thereof which binds to the extracellular domain of Patched-1. In some aspects, the epitope binding agent can comprise isolated antibody. In some aspects, the epitope binding agent can comprise at least an epitope binding fragment of the antibody disclosed herein. In some aspects, the epitope binding agent comprising at least an epitope binding fragment of one or more of the antibodies disclosed herein may be further conjugated with other molecules as disclosed herein.

An antibody or epitope binding agent “which binds” an antigen of interest, e.g., Patched-1 protein, is one that binds the antigen with sufficient affinity such that the antibody/epitope binding agent is useful as an assay reagent, e.g., as a capture or as a detection antibody/epitope binding agent. Typically, such an antibody/epitope binding agent does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. An antibody that “specifically binds” to an antigen or an epitope is a term well understood in the art. A molecule is said to exhibit “specific binding” if for example it reacts more frequently, with more avidity, more rapidly, with greater duration, and/or with greater affinity with a particular target antigen than it does with alternative targets. As an example, an antibody that specifically (or preferentially) binds to an antigen (e.g., Patched-1) or an antigenic epitope therein is an antibody that binds this target antigen with greater affinity, avidity, more readily, and/or with greater duration than it binds to other antigens or other epitopes in the same antigen. Specific binding can be measured, for example, by determining binding of a target molecule compared to binding of a control molecule, which generally is a molecule of similar structure that does not have binding activity.

As used herein, “binding affinity” refers to the apparent association constant or KA. The KA is the reciprocal of the dissociation constant (K_{D}).

A “capture antibody,” as used herein, refers to an antibody that specifically binds a target molecule, e.g., a form of Patched-1, in a sample. Under certain conditions, the capture antibody forms a complex with the target molecule such that the antibody-target molecule complex can be separated from the rest of the sample. In certain aspects, such separation may include washing away substances or material in the sample that did not bind the capture antibody. In certain aspects, a capture antibody may be attached to a solid support surface, such as, for example but not limited to, a plate or a bead, e.g., a paramagnetic bead. In some aspects the capture antibody may be conjugated to a detection molecule or label. Examples of detection labels and conjugates are provided herein throughout the application.

A “detection antibody,” as used herein, refers to an antibody that specifically binds a target molecule in a sample or in a sample-capture antibody combination material. Under certain conditions, the detection antibody forms a complex with the target molecule or with a target molecule-capture antibody complex. A detection antibody is capable of being detected either directly through a detection molecule or label or dye, which may be amplified, or indirectly, e.g., through use of another antibody that is labeled and that binds the detection antibody. For direct labeling, the detection antibody is typically conjugated to a moiety that is detectable by some means, for example, including but not limited to, biotin, horse radish peroxidase (HRP), fluorescent molecules, radioactive isotopes etc. Other examples of detection labels and conjugates are provided herein.

The term “chimeric” antibody refers to an antibody in which a portion of the heavy and/or light chain is derived from a particular source or species, while the remainder of the heavy and/or light chain is derived from a different source or species.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hyper-variable regions (CDRs). See, e.g., Kindt et al. Kuby Immunology, 6th ed., W.H. Freeman and Co., page 91 (2007), the disclosure of which is incorporated in its entirety herein. A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J Immunol. 150:880-887 (1993); Clarkson et al., Nature 352:624-628 (1991), the disclosures of which are incorporated in their entirety herein.

The terms “host cell,” “host cell line,” and “host cell culture” as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein. In some aspects the host cell may be a hybridoma.

The terms “label” or “detectable label,” or “detection molecule” or “detectible molecule” or “conjugate” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label may signal its presence by participating in specific binding reactions. Non-limiting examples of such labels include haptens, antibodies, biotin, streptavidin, his-tag, nitrilotriacetic acid, glutathione S-transferase, glutathione and the like. Further specific examples are provided in the current disclosure.

The term “detection means” as used herein, refers to a moiety or technique used to detect the presence of the detectable antibody through signal reporting that is then read out in an assay. Typically, a detection means employ reagents, e.g., a detection agent, that amplify an immobilized label such as the label captured onto a microtiter plate, e.g., avidin, streptavidin-HRP or streptavidin-β-D-galactopyranose.

The term “detecting,” is used herein, to include both qualitative and quantitative measurements of a target molecule, e.g., Patched-1 or processed forms thereof. In certain aspects, detecting includes identifying the mere presence of the target molecule in a sample as well as deter-mining whether the target molecule is present in the sample at detectable levels.

The term “sample” is used herein to encompass both biological and non-biological samples. In some aspects the sample may be a clinical sample. In some aspects the sample may be a non-clinical sample. In some aspects the sample may be a laboratory sample. The term sample includes both fluid and non-fluid samples. Examples of fluid samples include but are not limited to biological fluids, cell lysates, non-biological fluids like buffers etc. Non-limiting sources of a biological sample for use in the present disclosure include cells, solid tissue, biopsy, ascites, aspirates, fluidic extracts, blood (including circulating cells), plasma, serum, spinal fluid, lymph fluid, the external sections of the skin, respiratory, intestinal, and genitourinary tracts, tears, saliva, milk, tumors, organs, cell cultures and/or cell culture constituents, for example. Methods for obtaining tissue samples and body fluids from animals (e.g., humans) are well known in the art. Non-fluid samples include but are not limited to cells, tissue samples, samples bound to matrices, strips, solid substrate material or membrane (e.g., plastic, nylon, paper), plates etc.

An “individual” or “subject,” as used interchangeably herein, is a mammal. In certain aspects, the individual or subject is a human.

It should also be understood that, unless clearly indicated to the contrary, in any methods disclosed herein that include more than one step or act, the order of the steps or acts of the method is not necessarily limited to the order in which the steps or acts of the method are recited.

### II. Compositions

In some aspects the current disclosure encompasses compositions comprising isolated antibodies, epitope binding fragment, epitope binding agent, or variant and conjugates thereof and/or nucleic acid sequences encoding the disclosed antibodies, epitope binding fragment, epitope binding agent, or variant and conjugates thereof that specifically bind Patched-1.

### Antibodies and Epitope Binding Agents

In some aspects the antibodies or epitope binding agents provided herein comprise an amino acid sequence that is at least about 80% identical to one or more of SEQ ID NOs: 1-6, or at least 60% identical to SEQ ID NOs: 7-15 provided in Table 1. In some aspects the antibody or epitope binding agent comprises an amino acid sequence that is at least about 80 to about 85%, or about 85% to about 90%, or about 90% to about 95%, or about 95% to about 100% identical to one or more of SEQ ID NOs: 1-6 or at least about 60 to about 65%, or about 65% to about 70%, or about 70% to about 75%, or about 75% to about 80% or about 80 to about 85%, or about 85% to about 90%, or about 90% to about 95%, or about 95% to about 100% identical to one or more of SEQ ID NOs: 7-15. In some exemplary aspects the epitope binding agents provided herein recognize certain sequence and/or structural features of folded human Patched-1. In some exemplary aspects the epitope binding agents provided herein recognize structural features of the extracellular domain of human Patched-1.

TABLE 1
Amino acid sequences

| SEQ ID | | |
|---|---|---|
| NO | Name | Sequence |
| 1 | V_{H}(6H3) | EVQLVESGGGLVKPGGSRKLSCVASGFTLSDYGMHWVRQA |
| | | PEKGLEWVAYIGSDSYTIYHADTMKGRFTISRDNAKNTLF |
| | | LQMTSLRSEDTAMYYCGRNYGMDYWGQGTS |
| 2 | V_{L}(6H3) | VLTQSPAIMSASLGERVTLTCTASSSVTSSYLHWYQQKPG |
| | | SSPKLWIYSTSNLPFGVSPRFSGSGSGTSFSLTISSMEAE |
| | | DAATYYCHQFHRSPYTFGGGTK |
| 3 | V_{H}(4G8) | EVQLVESGGGLVKPGGSRKLSCAASGFTLSDYGMHWVRQA |
| | | PDKGLEWVAYISSDSNTIYYADTVKGRFTISRDNARNTLF |
| | | LQMTSLRSEDTAIYYCTRNYGMDYWGQGTS |
| 4 | V_{L}(4G8) | VLTQSPAIMSASLGERVTMTCTASSSVSSTYLHWYQQNPG |
| | | SSPKLWIYSASNLASGVPTRFSGSGSGTSYSLTISSMEAE |
| | | DAATYYCHQFHRSPYTFGGGTK |
| 5 | V_{H}(4H2) | EVQLVESGGGLVKPGGSRKLSCAASGFTLSDYGMHWVRQA |
| | | PDKGLEWIAYISSDSNTIHYTDTVKGRFTISRDNARNTLF |
| | | LQMTSLRSEDTAMYYCTRNYGMDYWGQGTS |
| 6 | V_{L}(4H2) | VLTQSPAIMSASLGERVTMTCTASSSVSSTYLHWYQQNPG |
| | | SSPKLWIYSASNLASGVPTRFSGSGSGTSYSLTISSMEAE |
| | | DAATYYCHQFHRSPYTFGGGTK |
| 7 | HC-CDR1 | GFTLSDY |
| 8 | HC-CDR2 [6H3] | GSDSYT |
| 9 | HC-CDR2 [4G8, | SSDSNT |
| | 4H2] | |
| 10 | HC-CDR3 | NYGMDY |
| | (all are same) | |
| 11 | LC-CDR1 [6H3] | ASSSVTSSY |
| 12 | LC-CDR1 [4G8, | ASSSVSSTY |
| | 4H2] | |
| 13 | LC-CDR2 [6H3] | YSTSNL |
| 14 | LC-CDR2 [4H2, | YSASNL |
| | 4G8] | |
| 15 | LC-CDR3 | QFHRSPY |

In some exemplary aspects the epitope binding agent is an isolated antibody, or variants, conjugates or fragments thereof that specifically binds Patched-1. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length comprising the Fc and Fab regions) polyclonal or monoclonal antibodies, but also antigen-binding fragments thereof (such as Fab, Fab′, F(ab′)2, Fv), single-chain antibody (scFv), fusion proteins comprising an antibody portion (e.g., chimeric antigen receptor or CAR), humanized antibodies, chimeric antibodies, diabodies, single domain antibody, nanobody (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. Furthermore, the term relates to modified and/or altered antibody molecules, as well as to recombinantly or synthetically generated/synthesized antibodies. The term “antibody” also comprises bifunctional antibodies, trifunctional antibodies, chimeric antibodies or antibody-fusion proteins. An antibody encompassed herein may include an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. In some exemplary aspects, the antibody is an IgG.

In some aspects the term “antibody” as used herein also comprises recombinant antibodies. The term “recombinant antibody” includes all antibodies that are prepared, expressed, created or isolated by recombinant means. Recombinant antibodies are e.g. antibodies obtained by B-cell PCR, or antibodies isolated from an animal (e.g., a mouse) that is transgenic for human immunoglobulin genes, antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Recombinant rabbit antibodies as produced by B-cell PCR have variable and constant regions (if present) derived from rabbit germline immunoglobulin sequences. i.e., the direct result of B-cell PCR are the binding relevant fragments of an antibody, and the skilled artisan has no problem whatsoever to e.g. construe a full length antibody, a chimeric antibody, or whatever “antibody” that will be desired/required. In some aspects the current disclosure encompasses recombinant antibodies comprising the amino acid sequence provided in any one or more of SEQ ID NOs: 1-15.

An antibody molecule encompassed herein may comprise a heavy chain variable region (V_{H}) and a light chain variable region (V_{L}). The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} may be composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) U.S. Department of Health and Human Services, NIH Publication No. 91-3242; Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004), the disclosures of which are incorporated in their entirety herein.

In some aspects, the current disclosure encompasses antibodies that comprise an amino acid sequence that is at least about 80% identical to one of more of the sequences provided in Table 1. In some aspects the isolated antibody provided herein comprises a sequence that is at least 80%, or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to any one or more of SEQ ID NOs: 1-6, or at least 60%, or 61%, or 62%, or 63%, or 64%, or 65%, or 66%, or 67%, or 68%, or 69%, or 70%, or 71%, or 72%, or 73%, or 74%, or 75%, or 76%, or 77%, or 78%, or 79%, or 80%, or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to any one or more of SEQ ID NOs: 7-15. In some aspects the antibody herein may comprise a heavy chain variable region that comprises a sequence that has at least 80% identity to the sequence of any one or more of SEQ ID. NOs: 1, 3 or 5. In some aspects, the antibody herein may comprise a light chain variable region that comprises a sequence that has at least 80% identity to the sequence of any one of SEQ ID. NOs: 2, 4 or 6. In some aspects, the antibody herein may comprise a heavy chain CDR that has at least 60% identity to the sequence of any one or more of SEQ ID NOs: 7-10. In some aspects the antibody herein may comprise a light chain CDR that has at least 60% identity to the sequence of any one or more of SEQ ID NOs: 11-15. In some exemplary aspects, the current disclosure encompasses an antibody comprising a heavy chain variable region having the sequence of SEQ ID NO: 1 and a light chain variable region having the sequence of SEQ ID NO: 2 or a heavy chain variable region having the sequence of SEQ ID NO: 1 and a light chain variable region having the sequence of SEQ ID NO: 4, or a heavy chain variable region having the sequence of SEQ ID NO: 1 and a light chain variable region having the sequence of SEQ ID NO: 6, or a heavy chain variable region having the sequence of SEQ ID NO: 3 and a light chain variable region having the sequence of SEQ ID NO: 2, or a heavy chain variable region having the sequence of SEQ ID NO: 3 and a light chain variable region having the sequence of SEQ ID NO: 4, or a heavy chain variable region having the sequence of SEQ ID NO: 3 and a light chain variable region having the sequence of SEQ ID NO: 6, or a heavy chain variable region having the sequence of SEQ ID NO: 5 and a light chain variable region having the sequence of SEQ ID NO: 2, or a heavy chain variable region having the sequence of SEQ ID NO: 5 and a light chain variable region having the sequence of SEQ ID NO: 4, or a heavy chain variable region having the sequence of SEQ ID NO: 5 and a light chain variable region having the sequence of SEQ ID NO: 6. In some aspects the antibody herein may comprise a heavy chain CDR that has the sequence of SEQ ID NOs: 7-10. In some aspects the antibody herein may comprise a light chain CDR that has the sequence of any one or more of SEQ ID NOs: 11-15.

In an exemplary aspect the antibody provided herein is a monoclonal antibody selected from IgG-6H3, IgG-4H2 or IgG-4G8 or a conjugate or variant thereof that specifically binds one or more structural epitopes on the extracellular domain of folded Patched-1 protein. In certain aspects, epitope binding agents or antibodies described herein may specifically bind to a corresponding target antigen (e.g., Patched-1) or an epitope thereof. In some aspects the antibodies provided herein recognize certain sequence and structural features of folded human Patched-1. In some aspects the epitope binding agents provided herein recognize structural features of the extracellular domain of human Patched-1.

In certain aspects, antibodies described herein may have a suitable binding affinity for a target antigen (e.g. Patched-1). In some aspects, an antibody described herein may have a binding affinity (K_{D}) of at least about 1000 nM, at least about 100 nM, at least about 10 nM, at least about 1 nM, at least about 0.1 nM, or lower for an epitope of Patched-1. In some aspects, an antibody described herein may have a binding affinity (K_{D}) between about 1000 nM to about 0.1 nM (e.g., about 1000 nM, about 750 nM, about 500 nM, about 250 nM, about 100 nM, about 75 nM, about 50 nM, about 25 nM, about 10 nM, about 5 nM, about 1 nM, about 0.75 nM, about 0.5 nM, about 0.25 nM, about 0.1 nM) for Patched-1. In some aspects, an antibody described herein may have a binding affinity (K_{D}) between about 50 nM to about 40 nM (e.g., about 50 nM, about 49 nM, about 48 nM, about 47 nM, about 46 nM, about 45 nM, about 44 nM, about 43 nM, about 42 nM, about 41 nM, about 40 nM) for a epitope on Patched-1. In some aspects, an antibody described herein may have a binding affinity (KD) between about 50 nM to about 40 nM (e.g., about 50 nM, about 49 nM, about 48 nM, about 47 nM, about 46 nM, about 45 nM, about 44 nM, about 43 nM, about 42 nM, about 41 nM, about 40 nM) for Patched-1 protein. In some aspects, binding affinity (or binding specificity) can be determined by a variety of methods including equilibrium dialysis, equilibrium binding, gel filtration, ELISA, surface plasmon resonance, and/or spectroscopy (e.g., using a fluorescence assay).

In some aspects, the heavy chain of the antibody disclosed herein may further comprise a heavy chain constant region (CH) or a portion thereof (e.g., CH1, CH2, CH3, or a combination thereof). In some aspects, a heavy chain constant region for use herein may be of any suitable origin, e.g., human, mouse, rat, or rabbit. In some aspects, alternatively or in addition, a light chain of any of the antibodies disclosed herein may further comprise a light chain constant region (CL), which can be any CL known in the art. In some aspects, a CL may be a kappa light chain. In some aspects, a CL may be a lambda light chain. Antibody heavy and light chain constant regions are well known in the art, e.g., those provided in the IMGT database (www.imgt.org) or at www.vbase2.org/vbstat.php., both of which are incorporated by reference herein.

In some aspects, the antibody may be a full-length antibody or an antigen-binding fragment thereof. In some aspects, isolated antibodies herein may be a full-length antibody, which is an IgG molecule. In some aspects, isolated antibodies herein may be a Fab, a (Fab′)2, and/or a single-chain antibody. In some aspects, antibodies disclosed herein may be a single chain antibody (scFv). In some aspects, scFv antibody herein may comprise a V_{H} fragment and a V_{L} fragment, which may be linked via a linker. In accordance with these aspects, a linker incorporated between the two variable regions herein may be a flexible linker, a rigid linker, a cleavable linker, or any combination thereof. In some aspects, a linker incorporated between the two variable regions herein may be a flexible peptide linker, a rigid peptide linker, a cleavable peptide linker, or any combination thereof. In accordance with these aspects, a peptide linker incorporated between the two variable regions herein may be at least one amino acid. In some aspects, a peptide linker incorporated between the two variable regions herein may be about 1 amino acid to about 50 amino acids (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 32, about 34, about 36, about 38, about 40, about 42, about 44, about 46, about 48, about 50). In some aspects, a scFv antibody herein may comprise a V_{H} fragment and a V_{L} fragment, which may be linked via a flexible peptide linker. In some exemplary aspects the antibody herein may comprise a heavy chain variable region having the amino acid sequence of any one of SEQ ID NOs: 1-3 and/or a light chain variable region having the sequence of any one of SEQ ID NOs: 4-6 and a linker.

In some aspects, a scFv antibody herein may be in the V_{H}→V_{L} orientation (from N-terminus to C-terminus). In some aspects, a scFv antibody herein may be in the V_{L}→V_{H} orientation (from N-terminus to C-terminus).

In some aspects, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, cryo-EM, competition assays, gene fragment expression assays, and synthetic peptide-based assays. In some aspects, epitope mapping can be used to determine the sequence, to which an antibody bind. In some particular aspects of the current disclosure the epitope comprises structural features of the extracellular domain of Patched-1.

In some aspects the current disclosure also encompasses epitope binding agent or antibodies disclosed herein conjugated to a detector molecule (labels, dyes, assay molecules) for example fluorescent labels, phosphorescent molecules, chemiluminescent molecules, chromophores, luminescent molecules, photoaffinity molecules, colored particles and/or ligands, such as biotin fluorescent dyes, electrochemiluminescence dyes, metal-chelate complexes or labels.

Examples of fluorescent dyes are described by Briggs et al “Synthesis of Functionalized Fluorescent Dyes and Their Coupling to Amines and Amino Acids,” J. Chem. Soc., Perkin-Trans. 1 (1997) 1051-1058, the disclosure of which is incorporated in its entirety herein. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

Luminescent dyes or labels can be further subcategorized into chemiluminescent and electro-chemiluminescent dyes. The different classes of chemiluminogenic labels include luminol, acridinium compounds, coelenterazine and analogues, dioxetanes, systems based on peroxyoxalic acid and their derivatives.

The labels of major relevance used as electrochemiluminescent labels are the Ruthenium- and the Iridium-based electrochemiluminescent complexes, respectively.

Electrochemiluminescence (ECL) is very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)₃]²⁺ (which releases a photon at ˜620 nm) regenerating with TPA (Tripropylamine) in liquid phase or liquid-solid interface are used as ECL-labels. Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescence is triggered by a voltage imposed on a working electrode at a particular time and in a particular manner. The light produced by the label is measured and indicates the presence or quantity of the analyte. Recently also Iridium-based ECL-labels have been described.

In one aspect the directly detectable label/molecule is a chemiluminescent or an electrochemiluminescent label. The light produced by the label is measured and directly or indirectly indicates the presence or quantity of the analyte.

Radioactive labels make use of radioisotopes (radionuclides), such as iodine (¹²⁵I, ¹²¹I, ¹²⁴I, ¹³¹I), carbon (¹⁴C, ¹¹C), sulfur (³⁵S), tritium (³H), indium (¹²¹In), Flourine (¹⁸F), Phosphorus (³²P), Copper (⁶⁴Cu), Gallium (⁶⁸Gn), Yittrium (⁸⁶Y), Zirconium (⁸⁹Zr), Technetium (⁹⁹TC), Indum (¹¹¹In), Xenon (¹³³Xe), Lutetium (¹⁷⁷Lu), or Astatine (²¹¹At).

In some aspects, the label is a metal-chelate complex. Metal-chelate complexes suitable as labels for imaging purposes are well-known in the art.

In other aspects, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity. Site-specific attachment of effector or reporter molecules, wherein the reporter or effector molecule is conjugated to a carbohydrate residue in the Fc region, has also been disclosed in the literature.

In some aspects the epitope binding agent or antibodies provided herein may also be conjugated to a polynucleotide or a nucleic acid. In some aspects the polynucleotide may be a DNA. In some aspects the polynucleotide may be an RNA. In some aspects the polynucleotide may be a modified DNA or RNA. In some aspects the polynucleotide may comprise a radionuclide.

### Polynucleotides and Host Cells

Polynucleotides, vectors, and host cells can be used to prepare any one of the antibodies disclosed herein (e.g., an antibody that binds Patched-1) using recombinant technology, as exemplified herein. In some aspects the current disclosure also encompasses polynucleotide sequences that comprise a nucleic acid sequence encoding a polypeptide corresponding one or more of SEQ ID NOs: 1-6 or at least 80% identical to SEQ ID NOs: 1-6. In some aspects the current disclosure also encompasses polynucleotide sequences that comprise a nucleic acid sequence encoding a polypeptide corresponding one or more of SEQ ID NOs: 7-15 or at least 60% identical to SEQ ID NOs: 7-15. In some aspects the polynucleotide sequence may comprise a nucleic acid sequence that is at least about 60% (e.g., about 60%, or about 65%, or about 70%, or about 75%, about 80%, about 85%, about 90%, about 95%, about 98% or 100%) identical to the sequence of any one or more of SEQ ID NOs: 16-33 as provided in Table 2. In some aspects, the polynucleotide sequence encodes a V_{H} and comprises a nucleic acid sequence at least about 80% identical to any one of SEQ ID NOs: 28-30. In some aspects, the polynucleotide sequence encodes a V_{L} and comprises a nucleic acid sequence at least about 80% identical to any one of SEQ ID NOs: 31-33. In some aspects, the polynucleotide sequence comprises a nucleic acid sequence at least about 60% identical to any one of SEQ ID NOs: 16-21. In some aspects, the polynucleotide sequence comprises a nucleic acid sequence at least about 80% identical to any one of SEQ ID NOs: 22-27.

TABLE 2
Nucleic Acid Sequences

| SEQ ID | | |
|---|---|---|
| NO | Name | Sequence |
| 16 | nHC-CDR1 | GGATTCACTTTAAGCGACTAT |
| | [6H3] | |
| 17 | nHC-CDR1 | GGATTCACTCTCAGTGACTAT |
| | [4G8, 4H2] | |
| 18 | nHC-CDR2 | GGTAGTGACAGTTATACC |
| | 18 [6H3] | |
| 19 | nHC-CDR2 | AGTAGTGACAGTAATACC |
| | 19 [4G8, | |
| | 4H2] | |
| 20 | nHC-CDR3 | AACTATGGTATGGACTAC |
| | [4G8, 6H3] | |
| 21 | nHC-CDR3 | AACTATGGCATGGACTAT |
| | [4H2] | |
| 22 | nLC-CDR1 | GCCAGCTCAAGTGTAACTTCCAGTTAC |
| | [6H3] | |
| 23 | nLC-CDR1 | GCCAGCTCAAGTGTAAGTTCCACTTAT |
| | [4G8] | |
| 24 | nLC-CDR1 | GCCAGCTCAAGTGTAAGTTCCACTTAC |
| | [4H2] | |
| 25 | nLC-CDR2 | TATAGCACATCCAACCTG |
| | [6H3] | |
| 26 | nLC-CDR2 | TATAGCGCATCCAACCTG |
| | [4G8, 4H2] | |
| 27 | nLC-CDR3 | CAGTTTCATCGTTCCCCGTAC |
| 28 | V_{H}[6H3] | GAGGTGCAGCTGGTGGAGTCTGGGGGAGGCTTAGTGAAGCCTGG |
| | | AGGGTCCCGGAAACTCTCCTGTGTAGCCTCTGGATTCACTTTAA |
| | | GCGACTATGGAATGCACTGGGTCCGTCAGGCTCCAGAGAAGGGG |
| | | CTGGAGTGGGTTGCATACATTGGTAGTGACAGTTATACCATCTA |
| | | CCATGCAGACACAATGAAGGGCCGATTCACCATCTCCAGAGACA |
| | | ATGCCAAGAACACCCTGTTCCTGCAGATGACCAGTCTAAGGTCT |
| | | GAGGACACAGCCATGTATTACTGTGGAAGGAACTATGGTATGGA |
| | | CTACTGGGGTCAAGGAACCTCA |
| 29 | V_{H}[4G8] | GAGGTGCAGCTGGTGGAGTCTGGGGGAGGCTTAGTGAAGCCTGG |
| | | AGGGTCCCGGAAACTCTCCTGTGCAGCCTCTGGATTCACTCTCA |
| | | GTGACTATGGAATGCACTGGGTCCGTCAGGCTCCAGACAAGGGG |
| | | CTGGAGTGGGTTGCATACATTAGTAGTGACAGTAATACCATCTA |
| | | CTATGCAGACACAGTGAAGGGCCGATTCACCATCTCCAGAGACA |
| | | ATGCCAGGAACACCCTGTTCCTGCAAATGACCAGTCTAAGGTCT |
| | | GAGGACACAGCCATATATTACTGTACAAGGAACTATGGTATGGA |
| | | CTACTGGGGTCAAGGAACCTCA |
| 30 | V_{H}[4H2] | GAGGTGCAGCTGGTGGAGTCTGGGGGAGGCTTAGTGAAGCCTGG |
| | | AGGGTCCCGGAAACTCTCCTGTGCAGCCTCTGGATTCACTCTCA |
| | | GTGACTATGGAATGCACTGGGTCCGTCAGGCTCCAGACAAGGGG |
| | | CTGGAGTGGATTGCATACATTAGTAGTGACAGTAATACCATCCA |
| | | CTATACAGACACAGTGAAGGGCCGATTCACCATCTCCAGAGACA |
| | | ATGCCAGGAACACCCTGTTCCTGCAAATGACCAGTCTAAGGTCT |
| | | GAAGACACAGCCATGTATTACTGTACAAGGAACTATGGCATGGA |
| | | CTATTGGGGTCAAGGAACCTCA |
| 31 | V_{L}[6H3] | GTTCTCACCCAGTCTCCAGCAATCATGTCTGCATCTCTAGGGGA |
| | | ACGGGTCACCTTGACCTGCACTGCCAGCTCAAGTGTAACTTCCA |
| | | GTTACTTGCACTGGTACCAGCAGAAGCCAGGATCCTCCCCCAAA |
| | | CTCTGGATTTATAGCACATCCAACCTGCCTTTTGGAGTCTCACC |
| | | TCGCTTCAGTGGCAGTGGGTCTGGGACCTCTTTCTCTCTCACAA |
| | | TCAGCAGCATGGAGGCTGAAGACGCTGCCACTTATTACTGCCAC |
| | | CAGTTTCATCGTTCCCCGTACACGTTCGGAGGGGGGACCAAG |
| 32 | V_{L}[4G8] | GTTCTCACCCAGTCTCCAGCAATCATGTCTGCATCTCTAGGGGA |
| | | ACGGGTCACCATGACCTGCACTGCCAGCTCAAGTGTAAGTTCCA |
| | | CTTATTTGCACTGGTACCAGCAGAACCCAGGATCCTCCCCCAAA |
| | | CTCTGGATTTATAGCGCATCCAACCTGGCTTCTGGAGTCCCAAC |
| | | TCGTTTCAGTGGCAGTGGGTCTGGGACCTCTTACTCTCTCACAA |
| | | TCAGCAGCATGGAGGCTGAAGACGCTGCCACTTATTACTGCCAC |
| | | CAGTTTCATCGTTCCCCGTACACGTTCGGAGGGGGGACCAAG |
| 33 | V_{L}[4H2] | GTTCTCACCCATCTCCAGCAATCATGTCTGCATCTCTGGGGGAA |
| | | CGGGTCACCATGACCTGCACTGCCAGCTCAAGTGTAAGTTCCAC |
| | | TTACTTGCACTGGTACCAACAGAACCCAGGATOTTCCCCCAAAC |
| | | TCTGGATTTATAGCGCATCCAACCTGGCTTCTGGAGTCCCAACT |
| | | CGTTTCAGTGGCAGTGGGTCTGGGACCTCTTACTCTCTCACAAT |
| | | CAGCAGCATGGAGGCTGAAGACGCTGCCACTTATTACTGCCACC |
| | | AGTTTCATCGTTCCCCGTACACGTTCGGAGGGGGGACCAAG |

In certain aspects, nucleic acids (i.e., polynucleotides) encoding the heavy and light chain of an antibody as described herein can be cloned into one expression vector, each nucleotide sequence being in operable linkage to a suitable promoter. In some aspects, each of the polynucleotide sequences encoding the heavy chain and light chain may be in operable linkage to a distinct promoter. In some aspects, polynucleotide sequences encoding the heavy chain and the light chain may be in operable linkage with a single promoter, such that both heavy and light chains are expressed from the same promoter. In some aspects, when necessary, an internal ribosomal entry site (IRES) can be inserted between the heavy chain and light chain encoding sequences.

In certain aspects, the polynucleotide sequences encoding the heavy chain and/or light chain of the antibodies described herein may the same polynucleotide sequences encoding the heavy chain and/or light chain as any of the exemplary antibodies described herein (e.g., 6H3, 4G8, 4GH2 or in certain aspects, antibodies disclosed herein may share one or more polynucleotide sequences provided in Table 2.

In some aspects, genetically engineered antibodies such as single-chain antibodies can be produced via, e.g., conventional recombinant technology or any methods known in the art. In some aspects, polynucleotide encoding a monoclonal antibody specific to a target antigen (e.g. Patched-1) can be readily isolated and sequenced using conventional procedures (e.g., by using oligonucleotide probes that are capable of binding specifically to genes encoding the heavy and light chains of the monoclonal antibodies). Once isolated, the polynucleotide sequence can be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. In some aspects the polynucleotide can be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some aspects, genetically engineered antibodies, such as chimeric or hybrid antibodies; can be prepared that have the binding specificity of a target antigen. In some particular aspects, the current disclosure also encompasses hybridomas producing the epitope binding agents or antibodies described herein. In some exemplary aspects the current disclosure encompasses a hybridoma producing the IgG-6H3, IgG-4H2 or IgG-4G8 monoclonal antibody that specifically binds to epitopes on the extracellular domain of Patched-1. In some aspects the current disclosure encompasses a host cell comprising a polypeptide at least 80% identical to any one or more of SEQ ID NOs: 16-33. In some aspects the current disclosure encompasses a host cell comprising a nucleic acid sequence that is at least about 60% (e.g., about 60%, about 65%, about 70%, about 75%, about 80%, about 85%, about 90%, about 95%, about 98%, about 100%) identical to the sequence of any one or more of SEQ ID NOs: 16-33.

In some aspects, a single-chain antibody herein can be prepared via recombinant technology by linking a polynucleotide sequence coding for a heavy chain variable region and a nucleotide sequence coding for a light chain variable region. In some aspects, a linker may be incorporated between the two variable regions. In some aspects, techniques described to produce single chain antibodies can be adapted to produce a phage or yeast scFv library and scFv clones specific to Patched-1.

In some aspects, one or more vectors (e.g., expression vectors) having nucleic acids encoding any of the epitope binding agents or antibodies or fragments and variants described herein can be introduced into suitable host cells for producing the antibodies (for example a vector comprising a nucleic acid sequence encoding a polypeptide at least 80% similar to any one or more of SEQ ID NOs: 1-15). In some aspects the current disclosure encompasses vectors comprising a nucleic acid sequence that is at least about 60% (e.g., about 60%, about 65%, about 70%, about 75%, about 80%, about 85%, about 90%, about 95%, about 98%, about 100%) identical to the sequence of any one or more of SEQ ID NOs: 16-33. In some aspects, host cells can be cultured under suitable conditions for expression of the epitope binding agent, antibody or any polypeptide chain or variants thereof. In some aspects, epitope binding agents, antibodies or polypeptide chains, fragments or variants thereof can be recovered from the cultured cells (e.g., from the cells or the culture supernatant) via a conventional method, e.g., affinity purification. In some aspects, polypeptide chains of the antibody herein can be incubated under suitable conditions for a suitable period of time allowing for production of the antibody or functional epitope binding agents.

In certain aspects, standard molecular biology techniques can be used to prepare the recombinant expression vector, transfect the host cells, select for transformants, culture the host cells, and recover the epitope binding agent or antibodies from the culture medium. In some aspects, some antibodies can be isolated by affinity chromatography with a Protein A or Protein G coupled matrix.

### Pharmaceutical and Diagnostic Compositions

In certain aspects, provided herein are pharmaceutical compositions comprising one or more of the antibodies or epitope binding agents disclosed herein. In some aspects, pharmaceutical compositions herein may comprise one or more of the antibodies disclosed herein and at least one pharmaceutically acceptable carrier and/or excipient. Pharmaceutically acceptable carriers or excipients suitable for the compositions described herein are well known to one of skill in the art of use for preserving and delivering antibodies, antibody fragments, epitope binding agent to any mammalian subject including humans and other mammals.

In certain aspects, the pharmaceutical compositions can further include a non-specific innate immune response stimulator mixture or composition or other immunomodulatory agent for enhancing an immune response. In accordance with these aspects, the non-specific innate immune response stimulator can elicit both a cell-mediated immune response and a humoral immune response.

In certain aspects, the pharmaceutical compositions to be used in the present methods can include pharmaceutically acceptable carriers, excipients, and/or stabilizers in the form of lyophilized formulations or aqueous solutions. In some aspects, acceptable carriers, excipients, and/or stabilizers are nontoxic to recipients at the dosages and concentrations used, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG).

In some aspects, the pharmaceutical composition described herein can have liposomes containing the antibodies (or the encoding nucleic acids). In some aspects, liposomes for use herein can be generated by the reverse phase evaporation method with a lipid composition having phosphatidylcholine, cholesterol and PEG-derivatized phosphatidylethanolamine (PEG-PE). In some aspects, liposomes for use herein can be extruded through filters of defined pore size to yield liposomes with the desired diameter.

In some aspects, antibodies, or the encoding nucleic acid(s) herein, can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions.

In some aspects, the pharmaceutical compositions described herein can be formulated in sustained-release format. In some aspects, pharmaceutical compositions herein to be used for in vivo administration may be sterile. In some aspects, this can be readily accomplished by, for example, filtration through sterile filtration membranes. In some aspects, therapeutic antibody compositions herein can be placed into a container having a sterile access port, for example, an intravenous solution bag or vial having a stopper pierceable by a hypodermic injection needle.

In certain aspects, pharmaceutical compositions described herein can be in unit dosage forms such as tablets, pills, capsules, powders, granules, solutions or suspensions, or suppositories, for oral, parenteral, or rectal administration, or administration by inhalation or insufflation.

In some aspects, emulsion compositions herein can be those prepared by mixing an antibody with Intralipid™ or the components thereof (soybean oil, egg phospholipids, glycerol and water).

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to about 20% oil, for example, between about 5% and about 20%. The fat emulsion can comprise fat droplets between about 0.1 μm and about 1.0 μm, particularly about 0.1 μm and 0.5 μm, and have a pH in the range of about 5.5 to about 8.0.

In some aspects, pharmaceutical compositions herein for inhalation or intranasal administration may include solutions and suspensions in pharmaceutically acceptable, aqueous or organic solvents, or mixtures thereof, and powders. In some aspects, liquid or solid compositions herein can contain suitable pharmaceutically acceptable excipients as set out above. In other aspects, the compositions can be administered by the oral or nasal respiratory route for local or systemic effect.

In some aspects, compositions can be in sterile pharmaceutically acceptable solvents can be nebulized by use of gases. In some aspects, nebulized solutions herein can be breathed directly from the nebulizing device, or the nebulizing device can be attached to a face mask, tent or intermittent positive pressure breathing machine. In some aspects, solution, suspension or powder compositions herein can be administered, preferably orally or nasally, from devices which deliver the formulation in an appropriate manner.

In some aspects, concentrations of antibodies disclosed herein can be a predetermined concentration or a standard concentration. In some aspects, the antibodies disclosed herein can be in a concentration of about 1 milligrams/milliliters (mg/ml) to about 500 mg/ml, about 1 mg/ml to about 250 mg/ml, about 1 mg/ml to about 200 mg/ml, about 1 mg/ml to about 150 mg/ml, about 1 mg/ml to about 100 mg/ml, about 1 mg/ml to about 75 mg/ml, about 1 mg/ml to about 50 mg/ml, about 0.1 mg/ml to about 100 mg/ml, or other suitable concentration. In some aspects, the antibody is formulated to a concentration of about 30 mg/ml. In some aspects, the antibody is lyophilized. In some aspects, the antibody is diluted in a suitable solution to a suitable concentration prior to administration (e.g., in a therapeutic application described below).

In some aspects, pharmaceutical compositions described herein can further include one or more therapeutic agents and/or antibodies used for treating a Hedgehog signaling pathway related disease for example cancer. In some aspects, the pharmaceutical composition may further comprise one or more of a chemotherapeutic agents, a NSAID, antibiotics, painkillers, immune boosters, immunomodulatory compounds etc.

In some aspects, the current disclosure also encompasses compositions that comprise one or more of these epitope binding agents or antibodies for in vivo diagnostic use. In some aspects, these diagnostic compositions are used in conjunction with in vivo imaging techniques. In some exemplary aspects, epitope binding agents or antibodies may be coupled to dyes, fluorophores or radiolabels as provided above, but for in vivo imaging of Patched-1 or Patched-1 interacting proteins. Epitope binding agents of the disclosure may be administered neat to detect levels of Patched-1 in vivo in accordance with the present disclosure. More commonly, however, they are administered in the context of acceptable compositions, that contain effective amounts of one or more antibodies together with one or more other ingredients known to those skilled in the art for formulating compositions for in vivo use.

Additional ingredients useful in preparing these in vivo diagnostics in accordance with the present disclosure include, for example, carriers (e.g., in liquid or solid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Liquid diagnostic compositions preferably contain one or more monoclonal antibodies of the disclosure and one or more liquid carriers to form solutions, suspensions, emulsions, syrups, or pressurized compositions. An acceptable liquid carriers include, for example water, organic solvents, acceptable oils or fat, or combinations thereof. The liquid carrier can contain other suitable additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other acceptable propellant.

### III. Methods of Use

Patched-1 is a transmembrane protein that is a key player in regulating the Hh signaling pathway. Hedgehog (Hh) signaling governs embryogenesis and adult tissue homeostasis in mammals and other multicellular organisms. Whereas deficient Hh signaling leads to birth defects, unrestrained Hh signaling is implicated in human cancers. Antibodies to folded, extracellular domain of human Patched-1 were so far unavailable. The current disclosure provides antibodies and epitope binding agents that specifically recognize extracellular domain of Patched-1 protein. Provided herein are methods of using these antibodies and epitope binding agents for clinical application, especially in treatment of cancer and other disease that can results from abnormal Hh signaling domain signaling. Also provided herein are in vitro and in vivo use of these antibodies in diagnostics and for laboratory use.

### Therapeutic or Clinical Use

In certain aspects, provided herein are methods of treatment and prevention of cancer and other Hedgehog (Hh) signaling pathway related disorders. In some aspects, methods herein may comprise administering to a subject in need thereof an effective amount of one or more of the antibodies disclosed herein or one or more of the pharmaceutical compositions disclosed herein. Thus, an aspect of the present disclosure is a method for treating a subject in need thereof.

In some aspects, the current disclosure encompasses methods of treatment and prevention of cancer and other Hedgehog (Hh) signaling pathway related disorders, wherein the method comprises disrupting and/or blocking of the Patched-1-Hh ligand interaction in a subject in need thereof. Patched-1 is the primary receptor for Hh-N ligands. Unliganded Patched-1 inhibits Hh signaling, and this repression is released when Hh binds to Patched-1. Specifically, after Hh binding, Patched-1 releases its inhibition of the oncoprotein Smoothened (Smo), a polytopic membrane receptor that activates the Gli transcription factors to up-regulate Hh target genes. In some aspects, disruption of the Patched-1-Hh interaction can result in reigning the unrestrained Hh signaling implicated in multiple disease states. In some aspects, the compositions disclosed herein are operable in partially or completely blocking the interaction between Patched-1 and one or more Hh-ligands. This results in disruption of downstream Hh-signaling. In a related aspect, the current disclosure encompasses a method of abrogating and/or suppressing Hh signaling in a subject in need thereof. In some aspects, the compositions disclosed herein are capable of competing with the Hh-N ligand for Patched-1 binding. As provided herein, disrupted ligand interaction, results in unliganded Patched-1 bound to the antigen binding agent disclosed herein. Unliganded Patched-1 can inhibit or reduce unrestrained Hh-signaling thus providing suitable treatment option of Hh-related disorders.

In some aspects, methods herein may comprise administering to a subject in need thereof a pharmaceutical composition as disclosed here, wherein the subject is a human subject having, suspected of having, or at risk for a Hh signaling pathway related disorder. In some aspects, the disorder is a tumor or cancer. In some aspects, the cancer can be any tumor or cancer that occurs in (or is a metastatic cancer originating from) the bladder, breast, bone, cervix, muscle, brain and nervous system, endocrine system, endometrium, eye, lip, oral, liver, lung, gastrointestinal system (e.g., colon, rectal), genitourinary and gynecologic systems (e.g., cervix, ovary), head and neck, hematopoietic system, kidney, skin, pancreas, prostate, thyroid, bone, thoracic and respiratory system, or any other human tissue that has undergone a malignant transformation. In some aspects, the tumor is a solid tumor derived from any human cell. In some aspects, the tumor may be a sarcoma, carcinoma, or lymphoma. In some aspects, the tumor is a liquid tumor.

Non-limiting examples of cancers that may be treated with a method of the disclosure may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood).

In some aspects, methods herein may comprise administering to a subject in need thereof an effective amount of one or more of the antibodies disclosed herein or one or more of the pharmaceutical compositions disclosed herein together with other therapeutic interventions. In some aspects, the compositions disclosed herein can be administered before, after or along with additional therapeutics and treatment methods, non-limiting examples of which include chemotherapy and radiation therapy.

As used herein, “an effective amount” refers to the amount of each active agent required to confer therapeutic effect on the subject, either alone or in combination with one or more other active agents. Determination of whether an amount of the antibodies disclosed herein achieved a therapeutic effect would be evident to one of skill in the art. Effective amounts vary, as recognized by those skilled in the art, depending on the condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In some aspects, a maximum dose of the individual components or combinations thereof may be used, that is, the highest safe dose according to sound medical judgment.

In some aspects, empirical considerations, such as the half-life, generally will contribute to the determination of the dosage. In some aspects, antibodies herein that are compatible with the human immune system, such as humanized antibodies, may be used to prolong half-life of the antibody and/or to prevent the antibody from being attacked by the host's immune system. In some aspects, frequency of administration may be determined and adjusted over the course of therapy, and is generally, but not necessarily, based on treatment and/or suppression and/or amelioration and/or delay of a target disease/disorder. In some aspects, sustained continuous release formulations of an antibody herein may be appropriate. Various formulations and devices for achieving sustained release are known in the art.

In some aspects, dosages for an antibody as described herein may be determined empirically in individuals who have been given one or more administration(s) an antibody disclosed herein. In accordance with some aspects herein, individuals can be given incremental dosages of the antibody. To assess efficacy of the antibody, an indicator of the disease/disorder can be followed.

In some aspects, for administration of any of the antibodies described herein, an initial candidate dosage can be about 2 mg/kg. For the purpose of the present disclosure, a typical daily dosage may range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, treatment methods of the present disclosure may be sustained until a desired suppression of symptoms occurs and/or until sufficient therapeutic levels are achieved to alleviate a target disease or disorder, or a symptom thereof. In some aspects, a dosing regimen herein may comprise administering an initial dose of about 2 mg/kg antibody, followed by a weekly maintenance dose of about 1 mg/kg of antibody, or followed by a maintenance dose of about 1 mg/kg antibody every other week. In some aspects, other dosage regimens may be useful, depending on the pattern of pharmacokinetic decay that the practitioner wishes to achieve. In accordance with some aspects herein, dosing from one-four times a week is contemplated. In some aspects, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) antibody may be used. In some aspects, dosing frequency may be once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. In some aspects, the progress of this therapy can be easily monitored by conventional techniques and assays. In some aspects, a dosing regimen (including the disclosed antibody used) suitable for use herein can vary over time.

In some aspects, for an adult patient of normal weight, doses ranging from about 0.3 to about 5.00 mg/kg antibody may be administered. In some aspects, the dosage of the antibody described herein can be about 10 mg/kg. The particular dosage regimen, i.e., dose, timing and repetition, can depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific polypeptides (or compositions thereof) employed, the type and severity of the disease/disorder, whether the antibody is administered for preventive or therapeutic purposes, previous therapy, the patient's clinical history and response to the antibody, an/or the discretion of the attending physician. In some aspects, a clinician will administer one or more of the disclosed antibodies until a dosage is reached that achieves the desired result. Methods of determining whether a dosage resulted in the desired result would be evident to one of skill in the art. In some aspects, administration of one or more antibodies herein can be continuous or intermittent, depending, for example, upon the recipient's physiological condition, whether the purpose of the administration is therapeutic or prophylactic, and other factors known to skilled practitioners. In some aspects, administration of an antibody herein may be essentially continuous over a preselected period of time or may be in a series of spaced dose, e.g., either before, during, or after developing a target disease or disorder.

Conventional methods, known to those of ordinary skill in the art of medicine, can be used to administer one or more pharmaceutical compositions disclosed herein to a subject, depending upon the type of disease to be treated or the site of the disease. In some aspects, compositions herein can be administered via other conventional routes, e.g., The compositions described herein, may be administered to a patient through one or more of a subcutaneous, intradermal, a parenteral, oral, intraadiposal, intraarterial, intraarticular, intracranial, intradermal, intralesional, intramuscular, intranasal, intrapericardial, intraocular, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or and transdermal route intranodal, intramedulla, intramuscular, by intravenous or intralymphatic injection, or intraperitoneal routes. In some aspects, compositions herein can be administered to the subject via injectable depot routes of administration such as using 1-, 3-, or 6-month depot injectable or biodegradable materials and methods.

### Non-Therapeutic Use

In some aspects, the current disclosure also encompasses use of the disclosed antibodies for non-clinical or clinical diagnostic purposes. The ability of the antibody provided herein to recognize and bind Patched-1 in a structurally specific way, may be utilized in methods for detection of folded protein in in vitro or ex vivo samples and in vivo with suitable administration means.

Assaying for the expression of Patched-1 protein is intended to include detection, qualitative or quantitative measurement or estimation of the level of Patched-1 protein or variants or fragments thereof in a sample either directly (e.g., by determining or estimating absolute protein level) or relatively (e.g., by comparing to Patched-1 protein level in a second sample or standard). Patched-1 polypeptide expression level in the sample can be measured or estimated and compared to a standard Patched-1 protein level, the standard being determined from a second biological sample healthy individual or being determined by averaging levels from a population of samples that are not diseased. As will be appreciated in the art, once the “standard” Patched-1 polypeptide level is known, it can be used repeatedly as a standard for comparison.

Samples as used herein may vary depending on the application. For example, it may be a biological sample or a non-biological sample. The term non-biological sample may include synthetic peptides, buffers, non-clinical fluids, artificial antigen bound surfaces etc. The term “biological sample” includes any biological specimen obtained from an individual. Suitable samples for use in the present disclosure include, without limitation, tissue samples, biopsy, cells, sections, whole blood, plasma, serum, saliva, urine, stool (i.e., feces), tears, and any other bodily fluid. One skilled in the art will appreciate that samples can be diluted prior to the analysis of marker levels. The sample may be a fluid sample, a solid sample or a sample bound to a solid surface like matrices, beads, strips, solid substrate material or membrane (e.g., plastic, nylon, paper), plates etc.

The clinical and non-clinical methods of use provided herein share some common principles for detection, qualitative or quantitative measurement. In general, the epitope binding agent or antibody provided herein is contacted with a sample comprising Patched-1 to form a complex that is either directly detectible due to the presence of a detectible molecule or can be indirectly detected by a detection antibody. Methods to conduct these assays are well established in the art.

In some aspects the current disclosure encompasses methods of using the epitope binding agent and antibodies provided herein to detect and/or assay Patched-1 protein (for clinical and non-clinical purposes) in a sample using methods known to those of skill in the art, including immunoassays, such as immunohistochemistry (IHC), enzyme linked immunosorbent assay (ELISA), fluorescence-activated cell sorting (FACS), immunohistochemistry (IHC), immunoprecipitation, radioimmunoassays, electrochemiluminescence-based detection assays, magnetic immunoassays, lateral flow assays, and related techniques and Western blotting. Additional suitable immunoassays for detecting the target antigen in a sample will be apparent to those of skill in the art. Methods for performing these assays are known in the art.

In some aspects, anti-Patched-1 antibodies or antigen-binding fragments thereof described herein can carry a detectable molecule. When radioactive labels are used, currently available counting procedures known in the art may be utilized to identify and quantitate the specific binding of anti-Patched-1 antibody or antigen-binding protein or fragments thereof to Patched-1 (e.g., human Patched-1). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-Patched-1 antibody or antigen-binding protein of fragments thereof under conditions that allow for the formation of a complex between the antibody or antigen-binding protein or fragment thereof and Patched-1. Any complexes formed between the antibody or antigen-binding protein or fragment thereof and Patched-1 may be detected and compared in the sample and the control. In light of the specific binding of the antibodies or antigen-binding fragments thereof described herein for Patched-1, the antibodies or antigen-binding fragments thereof can be used to specifically detect for example Patched-1 expression, e.g., in whole cells, cell lysates, membrane extracts, on cell membranes, or in cytoplasm. The antibodies or antigen-binding fragments thereof described herein can also be used to purify Patched-1 via immunoaffinity purification.

The steps of various useful immunodetection methods have been described in the scientific literature. In general, the immunobinding methods include obtaining a sample, e.g., a sample suspected of comprising Patched-1, and contacting the sample with a first anti-Patched-1 antibody in accordance with the present disclosure under conditions effective to allow the formation of immunocomplexes.

Contacting the chosen biological sample with the antibody under effective conditions and for a period of time sufficient to allow the formation of immune complexes (primary immune complexes) generally comprises adding the antibody composition to the sample and incubating the mixture for a period of time sufficient for the antibodies to form immune complexes with, i.e., to specifically bind to, any Patched-1 present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as for example any of radioactive, fluorescent, biological and enzymatic tags. In some aspects, a secondary binding agent, such as a second antibody and/or a biotin/avidin ligand binding arrangement, may be used in accordance with methodologies known in the art.

In some aspects, the first antibody that becomes bound within the primary immune complexes may be detected by means of a second binding agent that has binding affinity for the antibody. In these cases, the second binding agent may be linked to a detectable label or detectable molecules provided herein. In some aspects, the second binding agent is an antibody, which may thus be termed a “secondary” antibody. The primary immune complexes for example Patched-1-epitope binding agent complex are contacted with the labeled, secondary binding agent, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding agent, such as an antibody, that has binding affinity for the antibody is used to form secondary immune complexes, as described above. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable molecule, allowing detection of the tertiary immune complexes thus formed. This system may provide for signal amplification if this is desired. Thus, in some aspects, any of the primary (epitope binding agent of the current disclosure), secondary or tertiary antibodies may be conjugated to a detectible molecule (examples of which are provided herein).

In some aspects, a biotinylated antibody or epitope binding agent is used to detect the target antigen(s), and a second step antibody is then used to detect the biotin attached to the complexed antibody. In that method the sample to be tested is first incubated in a solution comprising the first step antibody. If the target antigen is present, some of the antibody specifically binds to the antigen to form a biotinylated antibody/antigen complex. The anti-body/antigen complex is then amplified by incubation in successive solutions of streptavidin (or avidin) and biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a conjugate can be produced that is macroscopically visible.

In one aspect, immunohistochemistry (IHC) is used for immunological detection. Using IHC, detection of Patched-1 in a sample can be achieved by targeting a sample with a binding agent, e.g., an anti-Patched-1 antibody or antigen-binding fragment thereof. The binding agent can be linked, either directly or indirectly to a detectable label or can be detected by another binding agent that is linked, either directly or indirectly to a detectable label. In one aspect, 3,3′-diaminobenzidine (DAB) is used in the HIC assay to detect the primary antibody bound to Patched-1. In one aspect, the concentration of the anti-Patched-1 antibody or antigen-binding fragment thereof in the IHC assay is about 1 μg/ml to about 50 μg/ml. In one aspect, the concentration of the anti-Patched-1 antibody or antigen-binding fragment thereof in the IHC assay is about 1 μg/ml to about 20 μg/ml. In one aspect, the concentration of the anti-Patched-1 antibody or antigen-binding fragment thereof in the IHC assay is about 10 μg/ml.

IHC can be performed on cells, cell pellets, tissues, preparations from blood, plasma, serum, or lymph fluid, etc. In some aspects, the samples are fixed samples. In some aspects, the samples are paraffin embedded samples. In some aspects, the samples are formalin fixed and paraffin embedded samples.

In one aspect, flow cytometry is used for immunological detection. Thus, for example, the number of antibodies bound per cell (ABC) can be assessed using flow cytometry.

In some aspects, the current disclosure also encompasses methods of using the compositions provided herein for in vivo diagnostics applications. In some aspects the compositions provided herein are used in medical imaging of a subject, in need thereof. In some exemplary aspects the method encompasses administering to a subject an imaging effective amount of an in vivo diagnostic composition, wherein the composition comprises the epitope binding agent coupled to a detectable moiety for example a dye or a radiolabel. The localization and accumulation of the imaging agent is then detected. The localization and accumulation of the imaging agent may be detected by for example radionuclide imaging, radio scintigraphy, nuclear magnetic resonance imaging, computed tomography, positron emission tomography, computerized axial tomography, X-ray or magnetic resonance imaging method.

### VI. Kits

In some aspects, the detection methods and compositions of this disclosure can be provided in the form of a kit. In some aspects, such a kit comprises the antibody or an epitope binding agent or fragment thereof provided herein.

In an exemplary aspect the kit comprises the epitope binding agent disclosed herein; a reporter molecule that detects the Patched-1-antibody (epitope binding agent) complex, suitable reagents and instructions for use.

In some exemplary aspects the current disclosure also encompasses kits for IHC and related applications comprising: the epitope binding agent compositions provided herein, a reporter molecule that detects Patched-1-antibody complex (epitope binding agent), suitable detection reagents, and instructions for use.

In some exemplary aspects the current disclosure also encompasses diagnostic kits comprising means for obtaining a sample from a subject in need thereof; the epitope binding agent provided herein, a reporter molecule that detects Patched-1-antibody (epitope binding agent) complex, reagents and instructions for use.

In some aspects, such a kit is a packaged combination including the basic elements of: a capture antibody comprised of an anti-Patched-1 antibody; a detectable (labeled or unlabeled) antibody that binds to the antibody of interest and instructions on how to perform the assay method using these reagents.

The kit may further comprise a solid support for the capture reagents, which may be provided as a separate element or on which the capture reagents are already immobilized. In some aspect the capture antibody may already be immobilized on the solid support for example a plate, matrix, paper strip, plastic strip, beads.

Hence, the capture antibodies in the kit may be immobilized on a solid support, or they may be immobilized on such support that is included with the kit or provided separately from the kit. In some aspects, the capture reagents are coated on or attached to a solid material (for example, a microtiter plate, beads or a comb). The detectable antibodies may be labeled antibodies detected directly or unlabeled antibodies that are detected by labeled antibodies directed against the unlabeled antibodies raised in a different species or targeted to another epitope. Where the label is an enzyme, the kit will ordinarily include substrates and cofactors required by the enzyme; where the label is a fluorophore, a dye precursor that provides the detectable chromophore; and where the label is biotin, an avidin such as avidin, streptavidin, or streptavidin conjugated to HRP or β-galactosidase with MUG.

The components of the kit will be provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution will provide for a reagent solution having the appropriate concentration for combining with the sample to be tested.

The present disclosure also provides kits for use in treating or alleviating a target disease, such as cancer as described herein. Such kits can include one or more containers comprising an antibody, e.g., any of those described herein. In some aspects, an antibody herein may be co-used with a second therapeutic agent. In some aspects, the kits may further comprise suitable administration means like syringes, intravenous drip apparatus etc.

In some aspects the kits disclosed comprises a container and a label or package insert on or associated with the container. Suitable containers include, for example, bottles, vials, syringes, assay plates, strips, matrices etc. The containers may be formed from a variety of materials such as glass, plastic, paper etc. The kit may further include other materials desirable from a commercial and user standpoint, including other buffers, diluents, filters, needles, and syringes.

A “package insert” is used to refer to instructions customarily included in commercial packages of diagnostic products, that contain information about usage etc.

Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

Having described several aspects, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Example 1: Generation and Characterization of Anti-Human-Patched-1 Monoclonal Antibodies

Anti-human Patched-1 monoclonal antibodies were generated by immunizing NZB mice with Flag-tagged engineered human Patched-1 protein (with the deletion of residues 619-720, and 1189-1447, denoted as Ptch1*). Hybridomas were created by fusion of splenic B lymphocytes from hyperimmune mice to SP2-mIL6 mouse myeloma cells (CRL-2016). ELISA-positive, immunoblot-negative, immunoprecipitation-positive clones were serially diluted four times. The monoclonal hybridomas were expanded into a roller bottle culture and the culture supernatant purified on a Protein G Sepharose column by gravity flow. The purified antibodies were able to recognize folded human Patched-1 protein by immunoprecipitation. The variable regions were sequenced by RNA extraction (Qiagen), subsequent PCR (Superscript III) with degenerate primers. The resulting PCR sequences were analyzed with the IMGT database to determine complementarity-determining regions. A final PCR (Promega) with designed primers including restriction sites were used to clone possible nucleotide regions for the variable heavy and light regions into a shuttle vector with a His-tag and transfected into Expi293F cells (Thermo Fisher Scientific). The supernatant was collected and purified similarly on Ni-NTA gravity columns. The binding of the recombinant Fab regions was consistent with that of the antibodies produced by the hybridomas. The Fab regions could also be expressed in E. coli and the resulting proteins had comparable activities with these expressed in mammalian cells.

### Example 2: Structural and Functional Characterization of the Antibodies

### Methods

Patched-1 expression and purification: The construct of human Ptch1*was cloned into pEG BacMam with a C-terminal Flag-tag. The protein was expressed using baculovirus-mediated transduction of mammalian HEK-293S GnTI cells (ATCC). The cell lines tested negative for mycoplasma contamination. At 48 hours post-infection at 37° C., cells were disrupted by sonication in buffer A (20 mM Hepes pH 7.5, 150 mM NaCl) with 1 mM PMSF, 10 μg/mL leupeptin. After low-speed centrifugation, the resulting supernatant was incubated in buffer A with 1% (w/v) n-Dodecyl-β-D-maltoside (DDM, Anatrace) for 1 hour at 4° C. The lysate was centrifuged again, and the supernatant was loaded onto an Anti-Flag M2 affinity column (Sigma). After washing twice, the protein was eluted in buffer A with 0.1 mg/ml FLAG peptide, 0.02% DDM, and concentrated. The concentrated protein was purified by Superdex-200 size-exclusion chromatography (GE Healthcare) in buffer B (20 mM Hepes pH 7.5, 150 mM NaCl, and 0.06% (w/v) Digitonin (Sigma)). The peak fractions were collected for further experiments. Mass spectrometry (MS) and anti-Flag-tag Western blotting confirmed the identity of the protein.

Ptch1*-6H3^{Fab} complex assembly: For structure determination, the full-length 6H3 antibody purified from hybridoma medium was digested by papain protease (50:1, w/w) at 37° C. for 2 hours, followed by dialysis overnight and ion-exchange purification with a Hitrap Q column (GE Healthcare). Fractions containing the 6H3^{Fab} were collected and mix with purified Ptch1*protein at 1.5:1 molar ratio at 4° C. for 1 hour. The Ptch1*-6H3^{Fab} complex was purified by Superdex-200 size-exclusion chromatography (GE Healthcare) in buffer B. The peak fractions were collected and concentrated to 5-7 mg/ml for grid preparation.

EM Sample Preparation and Imaging: The freshly purified Ptch 1*-6H3^{Fab} complex samples were added to Glow discharged Quantifoil R1.2/1.3 400 mesh Au holey carbon grids (Quantifoil), blotted using a Vitrobot Mark IV (FEI), and frozen in liquid ethane. The grids were imaged in a 300 kV Titan Krios (FEI) with a Gatan K3 Summit direct electron detector (Gatan). Data were collected using SerialEM at 0.833 Å/pixel with a total dose of ˜60 electrons per Å2.

Imaging Processing and 3D reconstruction: Dark subtracted movie stacks were normalized by gain reference and the motion correction was performed using MotionCor2. The contrast transfer function (CTF) was estimated using CTFFIND. After particle picking by RELION-3, the low-quality images and false-positive particles were removed manually. After 2D-classification in CryoSPARC, classes with clean background were selected to generate initial models for the 3D-classification. Map from the best 3D class showing clear features of micelle, Fab were used as a model to fish out “good” particles from all the three data sets via 3D-classification. The resulting particles were subjected to the secondary 3D-classification in CryoSPARC. The best class was selected for the non-uniform refinement, CTF refinement and final local refinement in CryoSPARC.

Model Construction and refinement: Ptch1 structure from Ptch1-Shh-N complex (6E1H) and a structure of 6H3^{Fab} predicted by Swiss-Model were docked into the map as the initial model. Structure model were manually built by COOT, followed by refinement in real space using PHENIX and in reciprocal space using Refmac with secondary-structure restraints and stereochemical restraints.

Pull-Down Assay: For the pull-down assay, purified Ptch1*protein was immobilized to 20 μl Anti-Flag M2 resin, which was further incubated with antibodies 4G8, 4H2 and 6H3 for 1 hour at 4° C. Then the resin was spun down and washed 3 times with buffer B. The protein complex was eluted with 20 μl buffer B supplemented with 0.3 mg/ml FLAG peptide. 15 μl of the elution was loaded on SDS-PAGE for detection by Coomassie blue staining.

### Results

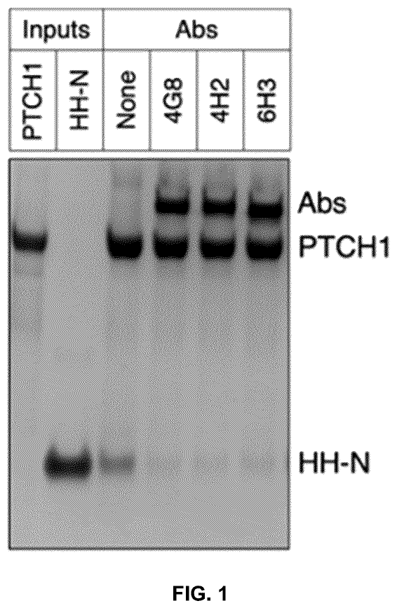

Studies were conducted to test if the disruption of the Patched-1-HH interaction can abrogate Hh signaling, thereby facilitating cancer therapy. Monoclonal antibodies 4G8, 4H2 and 6H3 raised as provided in Example 1 were used in a pull-down assay using Patched-1 or Hh-N as inputs. The antibodies were found to compete with Hh-N for Patched-1 binding in a pull-down assay (FIG. 1).

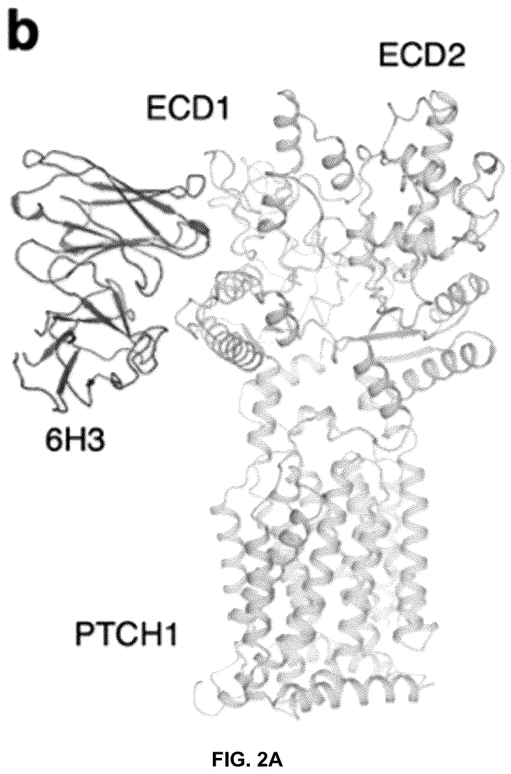

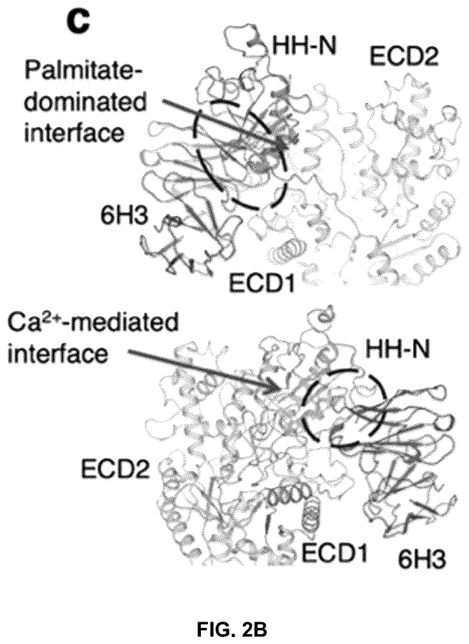

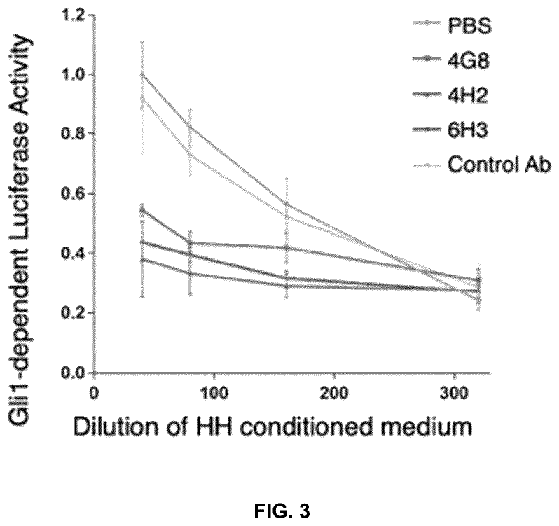

The structure of 6H3 in complex with Patched-1 demonstrates that the mAb binds to Patched-1's ECD1 and interferes with both Hh-N and Patched-1 interfaces. (FIGS. 2a and 2b). All three mAbs were able to suppress Hh signal in signaling assays (FIG. 3).

### Example 3: In Vivo Studies

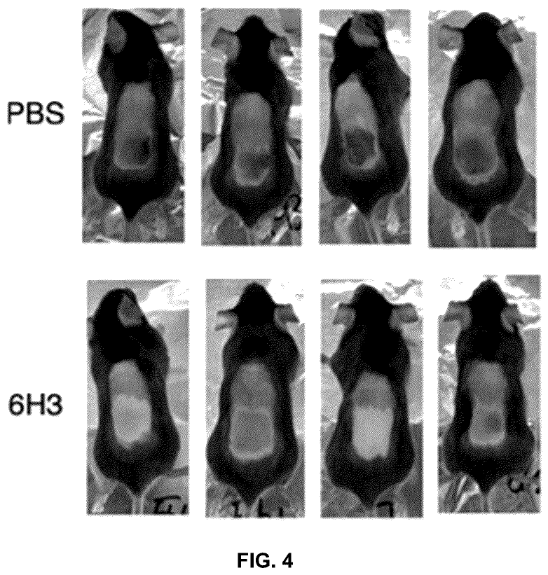

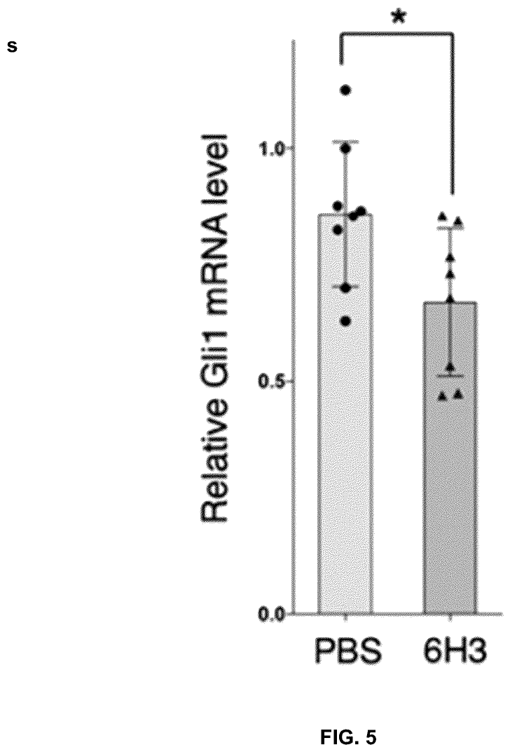

Next the inhibitory effect of 6H3 in mice was examined. Following a standard protocol to measure the Hh signaling activity at an animal level, 7-8 weeks old female C57BL/6 mice were shaved, and the bottom half of dorsal region were depilated with Nair. 6H3^{Fab} or PBS was injected (I.P., 20 mg/g) every other day for 2 weeks. The results indicated that 6H3 can suppress hair growth of mice with depilated dorsal hair (FIG. 4). In addition, the RNA was extracted from skin tissue to detect the mRNA level of Gli1 by qPCR. The results showed that Gli1 mRNA level was significantly lower in the skin tissue of 6H3-treated mice compared to the control group (FIG. 5). These results demonstrate that the mAbs developed herein inhibit Hh signaling both in vitro and in vivo by blocking the interaction between Patched-1 and Hh.

### REFERENCES

1 Qi, X. & Li, X. Mechanistic Insights into the Generation and Transduction of Hedgehog Signaling. Trends in biochemical sciences 45, 397-410, doi:10.1016/j.tibs.2020.01.006 (2020).
2 Chang, H. H. et al. Hedgehog overexpression leads to the formation of prostate cancer stem cells with metastatic property irrespective of androgen receptor expression in the mouse model. J Biomed Sci 18, 6, doi: 10.1186/1423-0127-18-6 (2011).
3 O'Toole, S. A. et al. Hedgehog overexpression is associated with stromal interactions and predicts for poor outcome in breast cancer. Cancer research 71, 4002-4014, doi: 10.1158/0008-5472.CAN-10-3738 (2011).
4 Niyaz, M., Khan, M. S., Wani, R. A., Shah, O. J. & Mudassar, S. Sonic Hedgehog Protein is Frequently Up-Regulated in Pancreatic Cancer Compared to Colorectal Cancer. Pathol Oncol Res 26, 551-557, doi: 10.1007/s12253-018-00564-2 (2020).
5 Qi, X., Schmiege, P., Coutavas, E., Wang, J. & Li, X. Structures of human Patched and its complex with native palmitoylated sonic hedgehog. Nature 560, 128-132, doi: 10.1038/s41586-018-0308-7 (2018).

1. An isolated antibody, epitope binding fragment or variant thereof, wherein the epitope binding fragment or variant thereof comprises one or more of:
(a) HC-CDR1 comprising the amino acid sequence at least 60% identical to the amino acid sequence set forth of SEQ ID NO: 7;
(b) HC-CDR2 comprising the amino acid sequence at least 60% identical to the amino acid sequence set forth of SEQ ID NO: 8 or SEQ ID NO: 9;
(c) HC-CDR3 comprising the amino acid sequence at least 60% identical to the amino acid sequence set forth of SEQ ID NO: 10;
(d) LC-CDR1 comprising the amino acid sequence at least 60% identical to the amino acid sequence set forth of SEQ ID NO: 11 or SEQ ID NO: 12;
(e) LC-CDR2 comprising the amino acid sequence at least 60% identical to the amino acid sequence set forth of SEQ ID NO: 13 or SEQ ID NO: 14, and
(f) LC-CDR3 comprising the amino acid sequence of any one of SEQ ID NO: 15.

2. The isolated antibody, epitope binding fragment, or variant thereof of claim 1 comprising:
(a) an immunoglobulin heavy chain variable region (V_{H}) that comprises at least 80% identity to the amino acid sequence set forth in SEQ ID NO: 1; and
(b) an immunoglobulin light chain variable region (V_{L}) that comprises at least 80% identity to the amino acid sequence set forth in SEQ ID NO: 2.

3. The isolated antibody, epitope binding fragment, or variant thereof of claim 1 comprising:
(a) an immunoglobulin heavy chain variable region (V_{H}) that comprises the amino acid sequence set forth in SEQ ID NOs: 1, 3, or 5; and
(b) an immunoglobulin light chain variable region (V_{L}) that comprises the amino acid sequence set forth in SEQ ID NOs: 2, 4, or 6;

4. The isolated antibody, epitope binding fragment or variant thereof of claim 1, wherein the V_{H} comprises an amino acid sequence comprising at least 80% identity to the amino acid sequence set forth in any one of SEQ ID NOs: 7-10.

5. The isolated antibody, epitope binding fragment or variant thereof of claim 1, wherein the V_{L} comprises an amino acid sequence comprising at least 80% identity to the amino acid sequence set forth in any one of SEQ ID NOs: 11-15.

6. The isolated antibody, epitope binding fragment or variant thereof of claim 1, wherein the antibody comprises:
a heavy chain variable region having the sequence of SEQ ID NO: 1 and a light chain variable region having the sequence of SEQ ID NO: 2;
a heavy chain variable region having the sequence of SEQ ID NO: 1 and a light chain variable region having the sequence of SEQ ID NO: 4;
a heavy chain variable region having the sequence of SEQ ID NO: 1 and a light chain variable region having the sequence of SEQ ID NO: 6;
a heavy chain variable region having the sequence of SEQ ID NO: 3 and a light chain variable region having the sequence of SEQ ID NO: 2;
a heavy chain variable region having the sequence of SEQ ID NO: 3 and a light chain variable region having the sequence of SEQ ID NO: 4;
a heavy chain variable region having the sequence of SEQ ID NO: 3 and a light chain variable region having the sequence of SEQ ID NO: 6;
a heavy chain variable region having the sequence of SEQ ID NO: 5 and a light chain variable region having the sequence of SEQ ID NO: 2;
a heavy chain variable region having the sequence of SEQ ID NO: 5 and a light chain variable region having the sequence of SEQ ID NO: 4; or
a heavy chain variable region having the sequence of SEQ ID NO: 5 and a light chain variable region having the sequence of SEQ ID NO: 6.

7. The isolated antibody, epitope binding fragment or variant thereof of claim 1, wherein the antibody is a full-length antibody.

8. The isolated antibody, epitope binding fragment or variant thereof of claim 7, wherein the antibody is any one of a monoclonal antibody, an IgG, Fv, single chain antibody, nanobody, diabody, scFv, Fab, F(ab′)2, and Fab.

9. The isolated antibody, epitope binding fragment or variant thereof of claim 8, wherein the antibody is a human or a humanized antibody.

10. The isolated antibody, epitope binding fragment or variant thereof of claim 1, further comprising a detection molecule.

11. The isolated antibody, epitope binding fragment or variant thereof of claim 10, wherein the detection molecule comprises a fluorescent label, phosphorescent it molecules, chemiluminescent molecules, radioactive isotope, chromophores, luminescent molecules, photoaffinity molecules, colored particles and/or ligands, such as biotin fluorescent dyes, electrochemiluminescence dyes, metal-chelate complexes or labels.

12. A pharmaceutical composition, comprising the isolated antibody, epitope binding fragment or variant thereof of claim 1, and a pharmaceutically acceptable carrier.

13. The pharmaceutical composition of claim 12, further comprising additional therapeutic agents.

14. The pharmaceutical composition of claim 13, wherein the additional therapeutic agent is a chemotherapeutic.

15. A polynucleotide sequence encoding an isolated antibody, epitope binding fragment or variant thereof of claim 1.

16. The polynucleotide sequence of claim 15, wherein the polynucleotide sequence comprises a nucleic acid sequence at least 60% identical to:
(a) nHC-CDR1 corresponding to any one of SEQ ID NOs: 16 or 17;
(b) nHC-CDR2 corresponding to any one of SEQ ID NOs: 18 or 19;
(c) nHC-CDR3 corresponding to any one of SEQ ID NOs: 20 or 21;
(d) nLC-CDR1 corresponding to any one of SEQ ID NOs: 22, 23, or 24;
(e) nLC-CDR2 corresponding to any one of SEQ ID NOs: 25 or 26; and
(f) nLC-CDR3 corresponding to any one of SEQ ID NO: 27.

17. The polynucleotide sequence of claim 15, encoding a variable heavy chain (V_{H}) comprising a nucleic acid sequence with at least 80% identity to any one of SEQ ID NOs: 28, 29, or 30.

18. The polynucleotide sequence of claim 15, encoding a variable light chain (V_{L}) comprising a nucleic acid sequence with at least 80% identity to of SEQ ID NOs: 31, 32, or 33.

19. A host cell comprising the polynucleotide sequence of claim 15.

20. A method of treatment or prophylaxis in a subject suffering from or suspected of hedgehog pathway related disease, the method comprising administering to the subject an effective amount of the pharmaceutical composition of claim 12.

21. A method of detecting Patched-1 on the surface of a cell, the method comprising:
(a) contacting the cell with the isolated antibody, epitope binding fragment or variant thereof of claim 1; wherein the isolated antibody, epitope binding fragment or variant thereof specifically binds to Patched-1 to comprise an antibody-Patched-1 complex;
(b) using a detector to determine presence of the antibody-Patched-1 complex; and
(c) quantifying the levels of Patched-1 on the cell.

22. The method of claim 21, further comprising a reporter molecule that specifically binds to the isolated antibody, epitope binding fragment or variant thereof.

23. A kit comprising:
(a) the isolated antibody, epitope binding fragment or variant thereof of claim 1;
(b) a reporter molecule that detects (a);
(c) reagents;
(d) instructions for use.


SEQUENCE LISTING (33 sequences)

SEQ ID NO: 1 - protein, 110 aa, synthetic construct
EVQLVESGGGLVKPGGSRKLSCVASGFTLSDYGMHWVRQAPEKGLEWVAYIGSDSYTIYHADTMKGRFTISRDNAKNTLFLQMTSLRSEDTAMYYCGRNYGMDYWGQGTS

SEQ ID NO: 2 - protein, 102 aa, synthetic construct
VLTQSPAIMSASLGERVTLTCTASSSVTSSYLHWYQQKPGSSPKLWIYSTSNLPFGVSPRFSGSGSGTSFSLTISSMEAEDAATYYCHQFHRSPYTFGGGTK

SEQ ID NO: 3 - protein, 110 aa, synthetic construct
EVQLVESGGGLVKPGGSRKLSCAASGFTLSDYGMHWVRQAPDKGLEWVAYISSDSNTIYYADTVKGRFTISRDNARNTLFLQMTSLRSEDTAIYYCTRNYGMDYWGQGTS

SEQ ID NO: 4 - protein, 102 aa, synthetic construct
VLTQSPAIMSASLGERVTMTCTASSSVSSTYLHWYQQNPGSSPKLWIYSASNLASGVPTRFSGSGSGTSYSLTISSMEAEDAATYYCHQFHRSPYTFGGGTK

SEQ ID NO: 5 - protein, 110 aa, synthetic construct
EVQLVESGGGLVKPGGSRKLSCAASGFTLSDYGMHWVRQAPDKGLEWIAYISSDSNTIHYTDTVKGRFTISRDNARNTLFLQMTSLRSEDTAMYYCTRNYGMDYWGQGTS

SEQ ID NO: 6 - protein, 102 aa, synthetic construct; identical to SEQ ID NO: 4

SEQ ID NO: 7 - protein, 7 aa, synthetic construct
GFTLSDY

SEQ ID NO: 8 - protein, 6 aa, synthetic construct
GSDSYT

SEQ ID NO: 9 - protein, 6 aa, synthetic construct
SSDSNT

SEQ ID NO: 10 - protein, 6 aa, synthetic construct
NYGMDY

SEQ ID NO: 11 - protein, 9 aa, synthetic construct
ASSSVTSSY

SEQ ID NO: 12 - protein, 9 aa, synthetic construct
ASSSVSSTY

SEQ ID NO: 13 - protein, 6 aa, synthetic construct
YSTSNL

SEQ ID NO: 14 - protein, 6 aa, synthetic construct
YSASNL

SEQ ID NO: 15 - protein, 7 aa, synthetic construct
QFHRSPY

SEQ ID NO: 16 - DNA, 21 bp, synthetic construct
  source 1..21: mol_type = other DNA
ggattcactttaagcgactat

SEQ ID NO: 17 - DNA, 21 bp, synthetic construct
  source 1..21: mol_type = other DNA
ggattcactctcagtgactat

SEQ ID NO: 18 - DNA, 18 bp, synthetic construct
  source 1..18: mol_type = other DNA
ggtagtgacagttatacc

SEQ ID NO: 19 - DNA, 18 bp, synthetic construct
  source 1..18: mol_type = other DNA
agtagtgacagtaatacc

SEQ ID NO: 20 - DNA, 18 bp, synthetic construct
  source 1..18: mol_type = other DNA
aactatggtatggactac

SEQ ID NO: 21 - DNA, 18 bp, synthetic construct
  source 1..18: mol_type = other DNA
aactatggcatggactat

SEQ ID NO: 22 - DNA, 27 bp, synthetic construct
  source 1..27: mol_type = other DNA
gccagctcaagtgtaacttccagttac

SEQ ID NO: 23 - DNA, 27 bp, synthetic construct
  source 1..27: mol_type = other DNA
gccagctcaagtgtaagttccacttat

SEQ ID NO: 24 - DNA, 27 bp, synthetic construct
  source 1..27: mol_type = other DNA
gccagctcaagtgtaagttccacttac

SEQ ID NO: 25 - DNA, 18 bp, synthetic construct
  source 1..18: mol_type = other DNA
tatagcacatccaacctg

SEQ ID NO: 26 - DNA, 18 bp, synthetic construct
  source 1..18: mol_type = other DNA
tatagcgcatccaacctg

SEQ ID NO: 27 - DNA, 21 bp, synthetic construct
  source 1..21: mol_type = other DNA
cagtttcatcgttccccgtac

SEQ ID NO: 28 - DNA, 330 bp, synthetic construct
  source 1..330: mol_type = other DNA
gaggtgcagctggtggagtctgggggaggcttagtgaagcctggagggtcccggaaactctcctgtgtagcctctggattcactttaagcgactatggaatgcactgggtccgtcaggctccagagaaggggctggagtgggttgcatacattggtagtgacagttataccatctaccatgcagacacaatgaagggccgattcaccatctccagagacaatgccaagaacaccctgttcctgcagatgaccagtctaaggtctgaggacacagccatgtattactgtggaaggaactatggtatggactactggggtcaaggaacctca

SEQ ID NO: 29 - DNA, 330 bp, synthetic construct
  source 1..330: mol_type = other DNA
gaggtgcagctggtggagtctgggggaggcttagtgaagcctggagggtcccggaaactctcctgtgcagcctctggattcactctcagtgactatggaatgcactgggtccgtcaggctccagacaaggggctggagtgggttgcatacattagtagtgacagtaataccatctactatgcagacacagtgaagggccgattcaccatctccagagacaatgccaggaacaccctgttcctgcaaatgaccagtctaaggtctgaggacacagccatatattactgtacaaggaactatggtatggactactggggtcaaggaacctca

SEQ ID NO: 30 - DNA, 330 bp, synthetic construct
  source 1..330: mol_type = other DNA
gaggtgcagctggtggagtctgggggaggcttagtgaagcctggagggtcccggaaactctcctgtgcagcctctggattcactctcagtgactatggaatgcactgggtccgtcaggctccagacaaggggctggagtggattgcatacattagtagtgacagtaataccatccactatacagacacagtgaagggccgattcaccatctccagagacaatgccaggaacaccctgttcctgcaaatgaccagtctaaggtctgaagacacagccatgtattactgtacaaggaactatggcatggactattggggtcaaggaacctca

SEQ ID NO: 31 - DNA, 306 bp, synthetic construct
  source 1..306: mol_type = other DNA
gttctcacccagtctccagcaatcatgtctgcatctctaggggaacgggtcaccttgacctgcactgccagctcaagtgtaacttccagttacttgcactggtaccagcagaagccaggatcctcccccaaactctggatttatagcacatccaacctgccttttggagtctcacctcgcttcagtggcagtgggtctgggacctctttctctctcacaatcagcagcatggaggctgaagacgctgccacttattactgccaccagtttcatcgttccccgtacacgttcggaggggggaccaag

SEQ ID NO: 32 - DNA, 306 bp, synthetic construct
  source 1..306: mol_type = other DNA
gttctcacccagtctccagcaatcatgtctgcatctctaggggaacgggtcaccatgacctgcactgccagctcaagtgtaagttccacttatttgcactggtaccagcagaacccaggatcctcccccaaactctggatttatagcgcatccaacctggcttctggagtcccaactcgtttcagtggcagtgggtctgggacctcttactctctcacaatcagcagcatggaggctgaagacgctgccacttattactgccaccagtttcatcgttccccgtacacgttcggaggggggaccaag

SEQ ID NO: 33 - DNA, 305 bp, synthetic construct
  source 1..305: mol_type = other DNA
gttctcacccatctccagcaatcatgtctgcatctctgggggaacgggtcaccatgacctgcactgccagctcaagtgtaagttccacttacttgcactggtaccaacagaacccaggatcttcccccaaactctggatttatagcgcatccaacctggcttctggagtcccaactcgtttcagtggcagtgggtctgggacctcttactctctcacaatcagcagcatggaggctgaagacgctgccacttattactgccaccagtttcatcgttccccgtacacgttcggaggggggaccaag